(19) United States
(12) Patent Application Publication
(10) Pub. No.: US 2022/0276222 A1
(43) Pub. Date: Sep. 1, 2022

(54) LIFECYCLE ASSESSMENT SYSTEMS AND METHODS FOR DETERMINING EMISSIONS AND CARBON CREDITS FROM PRODUCTION OF ANIMAL, CROP, ENERGY, MATERIAL, AND OTHER PRODUCTS

(71) Applicant: Low Carbon Leaf Beef, LLC, Glencoe, OK (US)

(72) Inventor: Colin M. Beal, Glencoe, OK (US)

(21) Appl. No.: 17/748,709

(22) Filed: May 19, 2022

Related U.S. Application Data
(63) Continuation-in-part of application No. 17/556,493, filed on Dec. 20, 2021.
Application No. 17/556,493 is a continuation of application No. 17/098,415, filed on Nov. 15, 2020, now Pat. No. 11,209,419.
(60) Provisional application No. 62/935,774, filed on Nov. 15, 2019.
(60) Provisional application No. 63/326,616, filed on Apr. 1, 2022.

(51) Int. Cl. G01N 33/497; G01N 33/00; G16B 20/00

(52) U.S. Cl. CPC G01N 33/497; G01N 33/0062; G01N 33/0031; G16B 20/00; G06F 30/27

(57) ABSTRACT

Approaches provide for machine learning or training algorithms that apply modifications to models based on a type of data obtained, including, for example, including, for example, producer-specific management practice data, performance data, energy production data, among other such data, to optimize models configured to quantify an amount of emissions emitted/generated by an emissions producing system. The emissions in certain embodiments can further enable the certification, label, or other transaction associated with emissions for individual animals, specifically identifiable crop products, specifically identifiable energy products, specifically identifiable materials, or other identifiable products.

### CROSS-REFERENCE TO RELATED APPLICATIONS

The present application is a continuation-in-part of U.S. patent application Ser. No. 17/556,493 filed Dec. 20, 2021, which is a continuation of U.S. patent application Ser. No. 17/098,415 filed Nov. 15, 2020, which claims priority to U.S. provisional application No. 62/935,774, filed Nov. 15, 2019, and entitled “LIFECYCLE ASSESSMENT SYSTEMS AND METHODS FOR DETERMINING ANIMAL GREENHOUSE GAS EMISSIONS,” which are hereby incorporated herein in their entirety for all purposes.

### BACKGROUND

As the global population increases and people desire more animal, crop, energy, material, and other products, demands for animal products (beef, chicken, pork, milk, eggs, etc.), crops (plants, algae, fungi, cyanobacteria, bacteria, etc.), energy carriers (oil, coal, gas, solar power electricity, wind power electricity, biofuels, etc.), materials (iron, stone, silver, gold, lithium, aluminum, woods, carbon dioxide, ammonia, propylene, etc.), and other products are expected to rise. This rise is expected to result in the rise of production of animal, crop, energy, material, and other products, as well as tradeoffs among these various products and the resources needed to produce them. For example: beef provides many nutritional benefits, but beef cattle can generate greenhouse gas (GHG) emissions; corn production provides nutrition for animals and people, as well as grain for biofuel production, but can generate GHG emissions, eutrophication emissions, and particulate matter emissions; oil-and-gas production provides valuable energy carries, but can generate GHG emissions, NOx emissions, and SOx emissions; lithium production yields valuable materials for batteries, but can generate emissions of H₂O, salt, H₂S, boron, etc.

These emissions can be generated from a variety of sources within the production lifecycles, including, for animals, for example: enteric methane production, methane and nitrous oxide from manure and fertilizer, direct emissions at the production site (such as diesel combustion in tractors, gas combustion in compressors, and a release of carbon stored in soil or water), and embedded upstream emissions generated during the production of fuels, feeds, seeds, fertilizers, chemicals, materials, etc. imported into the system. Meanwhile, as stated by some sources, global temperatures and atmospheric GHG concentrations continue to increase, raising concerns for some people about the negative impact of future climate changes on global society. As a result, there are sustainability initiatives aimed at reducing GHG emissions from segments of the economy, including, for example, transportation, electricity, industry, buildings segment, and agriculture.

Conventional lifecycle assessment (LCA) protocols have been developed for quantifying the environmental performance of a variety production systems for animal, crop, energy, material, and other products. However, many of these protocols provide performance results at the global, national, or aggregate producer level, without enabling the resolution to evaluate (and certify) performance of specifically identified animals, crop products, energy carriers, materials, or other products. As a result, some of these protocols have had limited practical market application and consumers have been unable to differentiate products produced with low emissions from those produced through conventional means with higher emissions.

### SUMMARY

Systems and methods in accordance with various embodiments describe model optimization techniques. In particular, various approaches describe machine learning or training algorithms that apply modifications to models based on a type of data obtained, including, for example, producer-specific management practice data, performance data, energy production data, among other such data, to optimize models configured to quantify an amount of emissions emitted/generated by an emissions producing system. The models can include, for example, product-centric models, animal-centric models, crop-centric models, energy production-centric models, a combination thereof, and the like. The emissions producing systems can include, for example, products derived from an animal emission producing system, a crop emission producing system, a product production emission producing system, a material production emission producing system, an energy production emission producing system, a combination thereof, and the like. A product can include, for example, animal products, crop products, energy products, material products, and the like.

The models can be configured to, for example, quantify emissions from production of animal, crop, energy, material, and other products (herein collectively also referred to as “emissions producing systems”).

In an example, a model can be configured to quantify emissions that an animal (cow, pig, chicken, etc.) may be expected to emit over a period of time, including, for example, over the animal's lifetime.

In another example, a model can be configured to quantify emissions that may be generated during the production of crops (corn, soy, wheat, algae, etc.), energy carriers, materials (e.g., plastic, iron, stone, graphite, graphene, ammonia, sulfuric acid, ethylene, propylene, lithium, silica/silicon, gold, diamonds, glass, etc.), or other products from the beginning to end of their production process.

In yet another example, a model can be configured to quantify emissions that may be generated during the production of energy.

In yet another example, a model can be configured to quantify emissions that may be generated during the production of material.

In yet another example, a model can be configured to quantify emissions from a product.

In yet another example, a model can be configured to quantify emissions from one or more (e.g., a combination of) emissions producing systems. For example, a model can be configured to quantify emissions from a crop producing system and an energy producing system, and/or segments from such systems. In a specific example, this can include configuring a model to quantify emissions from a derivative product. Said another way, a model configured to quantify the emissions from one product can be adapted to quantify the emissions from a derivative product. In a specific example, a model configured to quantify the emissions for a specific animal can be adapted to quantify the emissions for producing a piece a leather derived from the specific animal. In another example, a model used to quantify the emissions associated with producing a T-shirt includes the amount of emissions from a model configured to quantify the emissions from cotton production. In yet another example, a model used to quantify the emissions associated with producing plastic depends on the amount of emissions from a model configured to quantify the emissions from petroleum production.

In an embodiment, adapting the model can include, for example, incorporating emissions calculations from one or more product-specific and/or production-specific emissions models. In another example, adapting the model can include generating a model that includes appropriate equation components and adjusting (if needed) data variables associated with the equation components based on performance data or other such data.

In an embodiment, emissions can include emissions of any substance that can impact the environment, including but not limited to, greenhouse gas emissions such as CO₂, CH₄, N₂O, etc.; pollutants such as particulate matter, NOx, SOx, etc.; substances contributing to eutrophication and nutrients such as N, P, K, etc.; ozone depleting substances such as CFCs, etc.; toxicity substances such as herbicides, pesticides, anti-microbials, etc.; ionizing radiation such as U235, etc.; compounds such as H₂O, NaCl, H₂S, Cl₂, etc.; etc.

In an embodiment, data for use in determining gas emissions can be obtained, and a unique model (e.g., product-centric models, animal-centric models, crop-centric models, energy production-centric models, material-centric models, or a combination model) that utilizes the data can be generated. The data may include, for example, on-site practices management data, equipment management data, historic emissions and expected emissions data, expected progeny performance data, genetic data, phenotypic data, and/or animal, crop data, energy data, material data, or other product system data.

The data may be identified, extracted, and/or determined from a variety of different disaggregated sources. In an example, expected progeny performance data of animals can be used to estimate gas emissions (herein referred to as “emissions”) for select animals. Expected progeny performance data provides estimates of the genetic value of an animal as a parent. Essentially, differences in expected progeny performance data between animals of the same species may be used to predict differences in performance between their future offspring when each is mated to animals of the same average genetic merit. Various embodiments leverage expected progeny performance data to determine the emissions over an assessment cycle.

Genomic and genetic data can be used to, for example, determine the animal's or crop's genetic disposition to traits that influence emissions over an assessment cycle (e.g., feed efficiency, growth, yield, fertilizer requirements, water requirements, pest resistance, etc.). The genomic and genetic data generally refers to part of, or all of, an animal's sequenced genome including portions of the genome sometimes referred to as genetic markers, genetic sequences, single nucleotide polymorphisms (i.e., SNPs), DNA, DNA blocks, genes, or nucleotides. Genomic and genetic data may be used, alone or in combination with one or more other data and/or equations described herein to determine an animal's emissions or expected emissions over an assessment cycle.

Phenotypic data can be used to, for example, determine emissions over an assessment cycle. Phenotypic data generally refers to an animal's or crop's observable characteristics and production characteristics that can be measured, including, but not limited to: 1) measurements of body parts (e.g., meat, bone, hide, feet/hooves, feathers, head, leg, wing, muscles, udder, scrotum, etc.), plant parts (e.g., stem, ear, bean, cell, etc.) or the whole body or plant for heights, weights, lengths, colors, etc.; 2) measurements of body or plant parts, the whole body or plant, animal products (e.g., carcass, organs, tallow, milk, etc.), or waste products (e.g., manure, gases, shells, etc.) for composition data related to elements and compounds (C, N, P, CH₄, N₂O, etc.), biological components (proteins, amino acids, lipids, fatty acids, carbohydrates, water, etc.), economically valuable components (e.g., meat, bone, hide, prime cuts, omega-3 fatty acids, grains, beans, stover, etc.), etc.; 3) measurements of body or plant parts, the whole body or plant, animal or crop products, or waste products for yields of animal or crop products (e.g., eggs, milk, meat, cheese, blood, fat, protein, manure, corn grain, wheat bran, etc.), 4) measurements of body or plant parts, the whole body or plant, animal products, crop products, or waste products for yields of other biological or chemical data (e.g., rumen condition, milk somatic cell count, antibodies, moisture, mutations, etc.); 5) animal and crop performance data (e.g., behavior, docility, dry matter intake, energy intake, feed conversion efficiency, growth rate, water intake, reproduction/pregnancy rates, manure production, oocyte production, embryo production, color, density, etc.); 6) other measurable data related to livestock and crop production. An animal's or crop's measured phenotypic data may be used, alone or in combination with one or more other data and/or equations described herein, to determine an animal's emissions or expected emissions over an assessment cycle.

Physical, chemical, electrical, nuclear, magnetic, and thermal data, collectively called “property data” can be used to, for example, determine emissions over an assessment cycle. Property data generally refers to quantifiable characteristics of a material, substance, energy form, or energy carrier, such as, for example: mass, temperature, pressure, higher heating value, lower heating value, heat of combustion, heat content, energy content, metabolizable energy, density, energy density, melting point, conductivity, resistance, heat of vaporization, current, charge, voltage, electron volts, biochemical composition, protein content, amino acid profile, fat content, lipid content, fatty acid profile, fiber content, energy content, chemical bonds, moisture content, humidity, cellulose content, carbon content, nitrogen content, radiation, conduction, convection, photons, acoustics, Reynolds number, velocity, acceleration, matter, antimatter, or other quantifiable properties. Property data for animals, crops, energy, materials or other products may be used, alone or in combination with one or more other data and/or equations described herein, to determine an animal's emissions or expected emissions over an assessment cycle.

On-site practices and/or on-site management practices/protocol data can be used to, for example, determine emissions over an assessment cycle. For example, on-site practices management data refers to a variety of different data sources, including, but not limited to: feeds, fertilizers, herbicides, pesticides, manure management, grazing management, on on-site energy use, water supply (fresh water usage), etc.

On-site management practice data/protocols may be used, alone or in combination with one or more other data and/or equations described herein, to determine an animal, crop, energy, material or other product's emissions or expected emissions over an assessment cycle. Similar data may be obtained from on-site management and operations of oil-and-gas wells, coal mines, photovoltaic systems, wind power systems, refineries, biorefineries, transport systems, distribution systems, or other site-specific operations within a production system. For example, on-site data might be collected for methane leaks from a natural gas operation or carbon dioxide flue gas from a biorefinery. Similar data may be obtained from on-site management and operations of gold mines, lithium recovery ponds, direct air capture machines producing CO₂ or other gases, bentonite mines, propylene chemical plants, sodium hydroxide chemical plants, or other site-specific operations within a production system. For example, on-site data might be collected for energy consumption, GHG emissions from, and CO₂ collection by a direct air capture CO₂ plant.

In various embodiments, the data can be obtained using one or more sensors. For example, sensors can be used to monitor automatically and continuously: the consumption, emissions, and the behavior of animals; the growth, flux, and ecosystem impacts of crops; and the yield, properties, and environmental impacts of energy products. The data can be used to predict and determine a variety of conditions relating to health, performance, and production efficiency enabling determination of specific performance for identifiable products associated with different rations, response to medications, response to feed supplements, response to minerals and trace minerals, response to growth promoting substances, prediction of carcass quality, determination of greenhouse gas and manure excretion, fertilization, photosynthesis, evapotranspiration, growth rate, composition, gas flux, liquid flows, solid products, electrical components, etc.

Once the data is obtained, a unique model (e.g., a product-centric model, an animal-centric model, a crop-centric model, an energy production-centric model, a material-centric model, or combination thereof, etc.) that utilizes appropriate data can be generated. In an example, a crop-centric or crop model that utilizes crop data can be generated. In another example, an energy-system-centric or energy production model that utilizes the energy-specific data can be generated. In yet another example, a materials-system-centric or material production model that utilizes the material-specific data can be generated. In yet another example, a combination energy-system-centric and material production model can be generated.

Input parameters of the model(s) can be dynamically selected based on available data. In an example, one or more input parameters can be added or removed or otherwise selected based on the presence (or absence) of on-site practices management data, historic emissions and expected emissions data, properties data, genetic data, phenotypic data, property data, and/or obtained animal, crop, equipment, or system performance data. In short, the model can be dynamically updated based on available data, including updating the input parameters or weighting of those input parameters.

Thereafter, the model(s) can be used to determine emissions data for each emissions producing system. For example, the models can be used to determine emissions data for each animal or group of animals, crop or group of crop (e.g., one plant, a crop field, a microorganism culture volume, etc.), each energy carrier or group of energy carriers (e.g., an electron, a gallon of gasoline, a megajoule of heat, etc.), each material or group of materials (e.g., iron ore, lithium, water, ammonia, etc.), or other products.

The emissions data in certain embodiments can further enable the certification of emissions, emissions offsets (sometimes called credits), emissions limits (sometimes called caps), emissions taxes, emissions trades, and other emissions-related transactions for individual animals, specifically identifiable crop products (plant, algal, fungal, cyanobacterial, or bacterial crops), specifically identifiable energy products (physical, chemical, electrical, nuclear, etc.), specifically identifiable materials (organic, inorganic, metallic, stone, antimatter, etc.), or other identifiable products.

In some aspects, the techniques described herein relate to a computing system for generating emissions models, the computing system including: a computing device processor; and a memory device including instructions that, when executed by the computing device processor, enables the computing system to: obtain, by the computing device processor of the computing system, historic product data from a plurality of different disaggregated sources, identify, by the computing device processor of the computing system, a plurality of equation components based on the historic product data, individual equation components configured to quantify an amount of emissions, generate, by the computing device processor of the computing system, an emissions model including the plurality of equation components, the emissions model quantifying a total amount of emissions by a group of products for an emissions lifecycle of the group of products, wherein the emissions lifecycle includes a plurality of potential lifecycle emissions pathways, receive a selection of a product associated with the group of products to identify a selected product, the product associated with a unique identifier identifying the selected product, obtain in real-time from a database, by the computing device processor of the computing system, wherein the database is included of information obtained by at least one sensor of a plurality of sensors monitoring the selected product, performance data associated with the unique identifier of the selected product, identify, by the computing device processor of the computing system, one or more data variables associated with at least one equation component of the plurality of equation components of the emissions lifecycle based on the performance data, and apply, by the computing device processor of the computing system, at least one adjustment to the at least one equation component to generate a product-centric emissions model, the product-centric emissions model quantifying an amount of emissions by the selected product during an emissions assessment cycle of the selected product.

In some aspects, the techniques described herein relate to a computing system, wherein the instructions, when executed by the computing device processor, further enables the computing system to: receive a selection of a pathway from the plurality of potential assessment emissions pathways for the selected product, the pathway including an entry point corresponding to a start date and an exit point corresponding to an end date, wherein the product-centric emissions model is based on the pathway.

In some aspects, the techniques described herein relate to a computing system, wherein the instructions, when executed by the computing device processor, further enables the computing system to: identify, by the computing device processor of the computing system, equation components associated with the pathway, wherein the product-centric emissions model is based on the equation components.

In some aspects, the techniques described herein relate to a computing system, wherein the instructions, when executed by the computing device processor, further enables the computing system to: determine, by the computing device processor of the computing system, the amount of emissions by the selected product during the emissions assessment cycle of the selected product by evaluating the product-centric emissions model on the historic product data and the performance data.

In some aspects, the techniques described herein relate to a computing system, wherein the amount of emissions by the selected product is for a particular assessment emissions pathway.

In some aspects, the techniques described herein relate to a computing system, wherein the instructions, when executed by the computing device processor, further enables the computing system to: display, for the selected product associated with the unique identifier, in a graphical user interface one or more views of the amount of emissions during the emissions assessment cycle.

In some aspects, the techniques described herein relate to a computing system, wherein the instructions, when executed by the computing device processor, further enables the computing system to: associate at least one certification, label, emissions limit/cap, emissions trade, emissions offset/credit, or other emissions transaction with the selected product based on the amount of emissions by the selected product during the emissions assessment cycle of the selected product.

In some aspects, the techniques described herein relate to a computing system, wherein the at least one certification or other transaction indicates the amount of emissions that the product has emitted or is expected to emit.

In some aspects, the techniques described herein relate to a computing system, wherein the instructions, when executed by the computing device processor, further enables the computing system to: iteratively update the product-centric emissions model based on additional data from the plurality of sensors.

In some aspects, the techniques described herein relate to a computing system, wherein a machine learning technique is utilized to iteratively update the product-centric emissions model.

In some aspects, the techniques described herein relate to a computing system, wherein a machine learning technique is utilized to generate the emissions model.

In some aspects, the techniques described herein relate to a computing system, wherein the plurality of sensors includes at least one of a camera, a scale, a ruler, a timer, a feeder, a temperature sensor, a pressure sensor, a flow meter, an electrical sensor, a radiation sensor, a gas sensor, a liquid sensor, a humidity sensor, a movement sensor, a global positioning sensor (GPS), a soil composition sensor, a pH sensor, a body composition sensor, a health sensor, animal identification sensor, crop identification sensor, energy carrier identification sensor, material identification senso, facial identification sensor, biomedical sensor, an x-ray sensor, nuclear magnetic resonance sensor, or an ultrasound sensor, and wherein the performance data includes expected progeny performance data, expected progeny differences data, genetic data, phenotypic data, properties data and on-site practices management data associated with the selected product.

In some aspects, the techniques described herein relate to a computing system, wherein the instructions, when executed by the computing device processor, further enables the computing system to: generate control instructions to control an appliance to alter at least one task affecting the amount of emissions by the selected product during the emissions assessment cycle of the selected product.

In some aspects, the techniques described herein relate to a computer-implemented method for generating product-centric emissions models, including: obtaining, by a computing device processor, historic product data from a plurality of different disaggregated sources, identifying, by the computing device processor, a plurality of equation components based on the historic product data, individual equation components configured to quantify an amount of emissions, generating, by the computing device processor, an emissions model including the plurality of equation components, the emissions model quantifying a total amount of emissions by a group of products for an emissions assessment cycle of the group of products, wherein the emissions assessment cycle includes a plurality of potential assessment emissions pathways, receiving a selection of a product associated with the group of products to identify a selected product, the product associated with a unique identifier identifying the selected a product, obtaining in real-time from a database, by the computing device processor, wherein the database is included of information obtained by at least one sensor of a plurality of sensors monitoring the selected product, performance data associated with the unique identifier of the selected product, identifying, by the computing device processor, one or more data variables associated with at least one equation component of the plurality of equation components of the emissions assessment cycle based on the performance data, and applying, by the computing device processor, at least one adjustment to the at least one equation component to generate a product-centric emissions model, the product-centric emissions model quantifying an amount of emissions by the selected product during the emissions assessment cycle of the selected product.

In some aspects, the techniques described herein relate to a computer-implemented method, further including: receiving a selection of a pathway from the plurality of potential assessment emissions pathways for the selected product, the pathway including an entry point corresponding to a start date and an exit point corresponding to an end date, wherein the product-centric emissions model is based on the pathway.

In some aspects, the techniques described herein relate to a computer-implemented method, further including: identifying, by the computing device processor, equation components associated with the pathway, wherein the product-centric emissions model is based on the equation components.

In some aspects, the techniques described herein relate to a computer-implemented method, further including: determining, by the computing device processor, the amount of emissions by the selected product during the emissions assessment cycle of the selected product by evaluating the product-centric emissions model on the historic product data and the performance data.

In some aspects, the techniques described herein relate to a computer-implemented method, further including: determining, by the computing device processor, an emissions offset based on the total amount of emissions by the group of product and the amount of emissions by the selected product.

In some aspects, the techniques described herein relate to a computer-implemented method, wherein the database includes a blockchain database.

In some aspects, the techniques described herein relate to a non-transitory computer readable storage medium storing instructions that, when executed by a computing device processor of a computing system, causes the computing system to: obtain, by the computing device processor, historic product data from a plurality of different disaggregated sources, identify, by the computing device processor, a plurality of equation components based on the historic product data, individual equation components configured to quantify an amount of emissions, generate, by the computing device processor, an emissions model including the plurality of equation components, the emissions model quantifying a total amount of emissions by a group of products for an emissions assessment cycle of the group of products, wherein the emissions assessment cycle includes a plurality of potential assessment emissions pathways, receive a selection of a product associated with the group of products to identify a selected product, the product associated with a unique identifier identifying the selected product, obtain in real-time from a database, by the computing device processor, wherein the database is included of information obtained by at least one sensor of a plurality of sensors monitoring the selected product, performance data associated with the unique identifier of the selected product, identify, by the computing device processor, one or more data variables associated with at least one equation component of the plurality of equation components of the emissions assessment cycle based on the performance data, and apply, by the computing device processor, at least one adjustment to the at least one equation component to generate a product-centric emissions model, the product-centric emissions model quantifying an amount of emissions by the selected product during the emissions assessment cycle of the selected product.

Embodiments described herein primarily reference beef cattle. However, various embodiments may be applied to other livestock, including, but not limited to dairy cattle, poultry, swine, equine, etc. without departing from the scope of the embodiments described herein. Further, as described herein, various embodiments may be applied to crops, energy products and energy carriers, materials, and/or other products, etc. In an embodiment, crops can include, e.g., plants, algae, fungi, cyanobacteria, bacteria, microorganism, eukaryotes, prokaryotes, etc. that are produced for profit, subsistence, or during a particular cycle, without departing from the scope of the embodiments described herein. In an embodiment, energy products and energy carriers can include, e.g., electricity, liquid fuel, solid fuel, gaseous fuel, heat, hydrodynamic force, pressure, kinetic energy carries, chemical energy carriers, electrical energy carriers, thermal energy carriers, nuclear energy carriers, photons, electrons, acoustic energy, etc. that are produced for profit, subsistence, or during a particular cycle, without departing from the scope of the embodiments described herein. In an embodiment, materials and other products can include, e.g., minerals, metals, stones, compounds (gas, liquid, or solid), industrial chemicals, atmospheric materials, surface materials, subsurface materials, etc. that are produced for profit, subsistence, or during a particular cycle, without departing from the scope of the embodiments described herein.

Embodiments described herein primarily reference greenhouse gas emissions. However, various embodiments may be applied to other emissions, including, but not limited to pollutants, emissions causing eutrophication, ozone depleting substances, ionizing radiation, chemical compounds, etc. without departing from the scope of the embodiments described herein.

Embodiments provide a variety of advantages. For example, in accordance with various embodiments, with conventional approaches consumers do not have access to information regarding the production emissions of the animal, crop, energy, material and other products available for purchase. In accordance with various embodiments, a unique model based on available data including, e.g., performance data, genetic data, phenotypic data, property data and/or on-site practices management data, can be generated to determine a system's emissions. The data may be aggregated, and/or determined to generate a rating that is indicative of the amount of emissions a product may have emitted/generated or may be expected to emit/generate over its lifetime, “cradle-to-grave” period, or a certain start-and-end period (e.g., gate-to-gate, farm-to-table, well-to-wheel, entry-to-exit, etc.). The rating system may be used to inform consumers about greenhouse gas emissions that are associated with their consumption of animal, crop, energy, material, or other products. Moreover, the rating system may be used by on-site managers to raise animals in a more sustainable fashion to reduce the amount of emissions that are associated with raising and cultivating animals—or used analogously for crop, energy, material, or other product systems. Indeed, the ratings and/or some of the calculated output may be used to help production managers make better decisions about their practices to reduce negative emissions or increase positive emissions.

In addition to giving consumers more information regarding the emissions associated with individual products for purchase, various embodiments provide emissions information about the farms, facilities, refineries, and other operations where products were generated. This enhances consumer knowledge of emissions and allows the consumer to make informed purchasing decisions.

Additionally, various embodiments provide greater information about an animal or crop to agricultural producers and/or about energy or material products to energy or material producers. Agricultural producers analyze crop and livestock performance data and expected progeny performance data carefully when making breeding or planting decisions or selecting which organisms to purchase. Currently, crop and livestock performance data and expected progeny performance data do not contain emissions data for individual animals or specific crop products. By providing emissions data for individual animals, agricultural producers will have more information to use when making breeding or planting decisions or when selecting which organisms to purchase or reproduce. Similarly, energy producers analyze energy system performance data when making production decisions, such as for example whether to drill a production well or selecting which equipment to purchase/use. Currently, energy system performance data for emissions is not available for specific energy products. By providing emissions data for specific energy products, producers will have more information about which system to use. Similar improvements are available for materials and other products.

Another benefit in accordance with various embodiments is that approaches described herein encourage sustainable practices. Agricultural, energy, material and other producers that use this information to produce products with low emissions will mitigate emissions, and thereby institute more sustainable practices on their operations and through the entire supply chain. Such sustainable practices include using less fertilizer, being more thoughtful about water allocation and use, using more efficient equipment, using feeds and fuels that generate lower emissions, and reducing emissions from transport and retail storage.

Various other functions and advantages are described and suggested below as may be provided in accordance with the various embodiments.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1A illustrates a system for utilizing performance data of animals, crops, energy, materials or other product systems to estimate emissions for selected animals, crops, energy, materials, or other products in accordance with various embodiments.

FIG. 1B illustrates an example production system in accordance with various embodiments.

FIG. 2 illustrates an example model training pipeline that can be utilized in accordance with various embodiments.

FIG. 3 illustrates the emissions simulator in accordance with various embodiments.

FIG. 4A illustrates an example process for utilizing performance data of animals, crops, energy, materials, or other product systems to estimate emissions for selected products in accordance with an embodiment.

FIG. 4B illustrates an example process for utilizing performance data of crops, to estimate emissions for the production of selected crops in accordance with an embodiment.

FIG. 4C illustrates an example process for utilizing performance data of energy production, to estimate emissions for the production of energy production in accordance with an embodiment.

FIG. 4D illustrates an example process for utilizing performance data of product and material production, to estimate emissions for the production of products and material in accordance with an embodiment.

FIG. 5 illustrates an example process for updating a model using real-time data in accordance with various embodiments.

FIG. 6 illustrates an example process for updating a model based on lifecycle emissions pathways in accordance with various embodiments;

FIG. 7 illustrates an exemplary computing device that can be used in accordance with various embodiments.

FIG. 8 illustrates an exemplary standalone computing system that can be used in accordance with various embodiments.

FIG. 9 illustrates an embodiment of the computing architecture that can be used in accordance with various embodiments.

FIG. 10 illustrates an exemplary overview of a computer system that can be used in accordance with various embodiments.

### DETAILED DESCRIPTION

The inventive system and method (hereinafter sometimes referred to more simply as “system” or “method”) described herein uses historic data and performance data (hereinafter, performance data may more generally refer to expected progeny performance data, on-site practices management data, equipment management data, historic emissions and expected emissions data, properties data, genetic data, phenotypic data, and/or animal, crop data, energy data, material data, or other product system data) to trigger modifications to models based on a type of data obtained, where the model(s) can be used to determine estimated emissions for identifiable animals, crops, energy, materials, or other products, and/or a combination thereof. More succinctly, embodiments described herein describe systems and methods for measuring and tracking emissions from animal, crop, energy production, materials, or other products, and/or a combination thereof through the whole (or segment thereof) product life cycle using dynamically updating models. The system is a computer program product which collects information about selected products, and generates a model that utilizes the data and facilitates adjustments based on related variables, and then determines emissions data for each product or group of products. Specifically, various embodiments uniquely model a variety of performance data to determine a product's emissions. It also may certify an animal, crop, energy, material or other product's emissions based on its' own genomics and/or its' own performances. Approaches described herein can also adjust for on-site management protocols in its model to determine operations-specific emission rates.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step).

Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The figures described herein generally illustrate an example approaches to estimating emissions for selected products in accordance with various embodiments. It should be understood that reference numbers are carried over between figures for similar components for purposes of simplicity of explanation, but such usage should not be construed as a limitation on the various embodiments unless otherwise stated. It should be further noted that emissions can be emissions (i.e., discharge) of any substance that can impact the environment, including but not limited to greenhouse gas emissions such as CO₂, CH₄, N₂O, etc.; pollutants such as particulate matter, NOx, SOx, etc.; eutrophication and nutrients such as N, P, K, etc.; ozone depleting substances such as CFCs, etc.; toxicity substances such as herbicides, pesticides, anti-microbials, etc.; ironizing radiation such as U235, etc.; compounds such as H₂O, NaCl, CO₂, H₂S, Cl₂, etc.; etc. Reference to a product or products as cattle is merely an example, and embodiments described herein apply to any number of different types of animals, crops, energy, materials, or other products.

### Conceptual Architecture

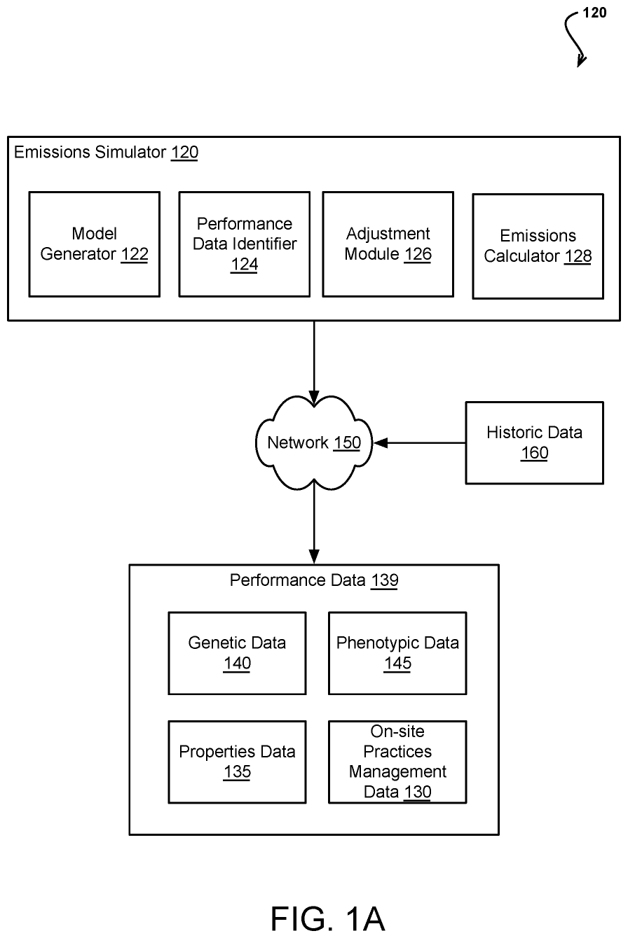

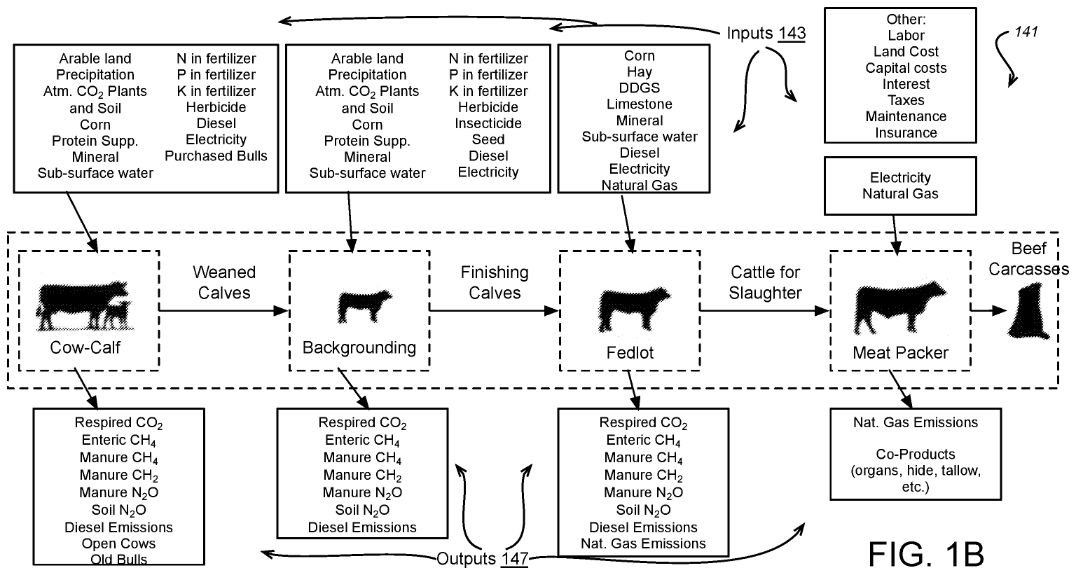

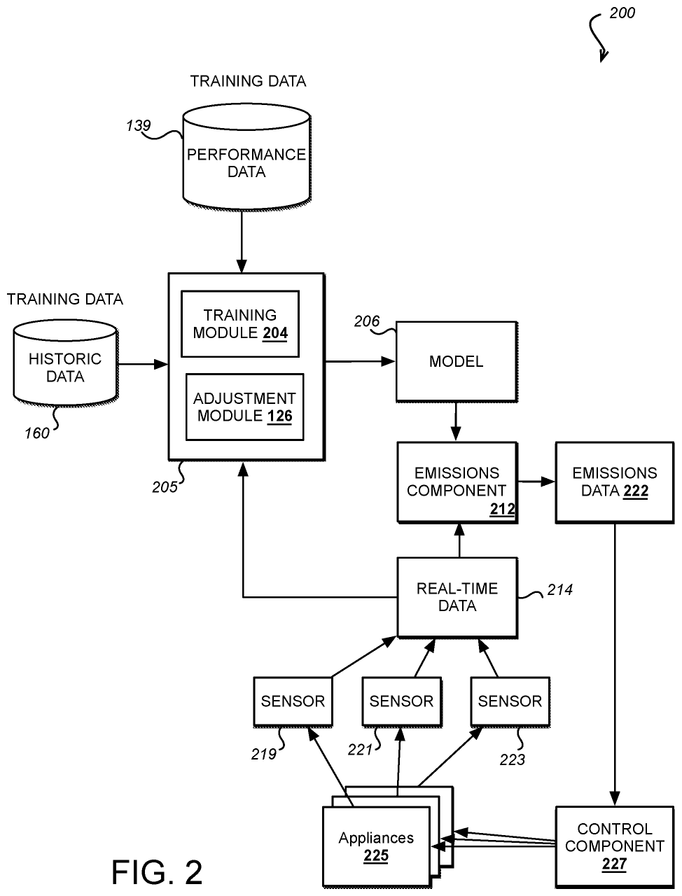

FIG. 1A illustrates a system for utilizing performance data of products to estimate emissions for selected products in accordance with various embodiments. The system is comprised of historic data 160, on-site practices management data 130, properties data 135, genetic data 140, phenotypic data 145, emissions simulator 120, and a network 150 over which the various systems communicate and interact. Emissions simulator 120 is described in greater detail in FIG. 2. Below, however, generally, emission simulator 120 generates a model that determines emissions.

As illustrated in FIG. 1A, the emissions simulator 120 may be comprised of model generator 122, performance data identifier 124, adjustment module 126, and emissions calculator 128. The various computing devices described herein are exemplary and for illustration purposes only. The system may be reorganized or consolidated, as understood by a person of ordinary skill in the art, to perform the same tasks on one or more other servers or computing devices without departing from the scope of embodiments described herein.

In an exemplary embodiment, the emissions simulator 120 obtains input parameters from historic data 160. Emissions simulator 120 generates a model, which includes equation components based on input parameters that model the impact of certain characteristics on a product's lifecycle emissions. Emissions simulator 120 obtains performance data 139, which may be comprised of properties data 135, genetic data 140, phenotypic data 145, and on-site practices management data 130, and identifies performance data variables (e.g., properties data variables, genetic data variables, phenotypic data variables, on-site practices management data variables) that may affect emissions. Emissions simulator 120 can apply adjustments to the model equations based on the performance data. The adjustments can be with respect to baseline emissions. For example, the adjustments can be with respect to baseline emissions for a particular type of oil production. Specifically, the emissions simulator can determine emissions output by an oil production system by applying the adjusted model equations to obtain model output.

Historic data 160 includes input parameters that impact emissions. The historic data may be compiled from academic papers, scientific literature, lifecycle assessment inventory databases, trade publications, experimental data, etc. In one embodiment, the relevant papers may specifically study the effects of various input parameters on emissions. In an embodiment, the relevant papers may study animal characteristics data that may or may not specifically analyze or study animal characteristic data and its direct impact on emissions. For example, the historic data 160 may be comprised of information about different parts of an animal lifecycle, including, but not limited to performance data 139, emissions information, economic impact information, etc.

In an embodiment, historic data 160 may be comprised of information about crop production comprised of, for example, information about different parts of crop production system, including, but not limited to performance data 139, emissions information, economic impact information, etc. Specifically, historic data 160 for crops may include crop yield relationships with fertilizer, geographic production data, genetic impacts, pest management data, water requirements, etc.

Historic data 160 for crops may be received from a variety of different disaggregated sources and proprietary databases, or may be generated based on data from academic papers, scientific literature, lifecycle assessment inventory databases, trade publications, experimental data, etc. One or more model equations can be generated, identified, selected, etc., based on the crop historic data.

In yet another embodiment, historic data 160 of information about energy or material production comprised of, for example, information about different parts of energy or material production system, including, but not limited to performance data 139, emissions information, economic impact information, etc.

Specifically, historic data 160 for energy or materials may include production yields, energy input requirements, transport requirements, end-use combustion performance, etc.

Historic data 160 for energy and material products may be received from a variety of different sources and proprietary databases, or may be generated based on based on data from academic papers, scientific literature, lifecycle assessment inventory databases, trade publications, experimental data, etc. One or more model equations can be generated, identified, selected, etc., based on the energy or materials historic data.

In yet another embodiment, the historic data 160 may be comprised of, for example, dry matter intake (DMI) data.

In one embodiment, the historic data 160 may be comprised of, for example, data from publications such as reports and papers from the IPCC, FAO, EIA, IEA, USDA, DOE, UN, World Bank, WRI, ecoinvent, GREET, scientific literature, or trade organizations.

In one embodiment, historic data 160 may be comprised of, for example, data from reports and textbooks including: International Organization for Standardization (ISO iso.org); 2006 IPCC Guidelines for National Greenhouse Gas Inventories (and subsequent updates); FAO Tackling Climate Change Through Livestock report; Inventory of US Greenhouse Gas Emissions and Sinks, U.S. EPA, 2018; 2019 Sustainability Report, Tyson Foods, 2020; Parker D. et al., Agricultural energy consumption, biomass generation, Texas A&M, 1997; Field T. and Taylor R., Scientific Farm Animal Production, 2020; etc.

In an embodiment, the historic data 160 may be comprised of, for example, data from lifecycle assessment inventories including: Wernet, G., Bauer, C., Steubing, B., Reinhard, J., Moreno-Ruiz, E., and Weidema, B., 2016, the ecoinvent database version 3 (part I): overview and methodology, the International Journal of Life Cycle Assessment, [online] 21(9), pp. 1218-1230, available at: <http://link.springer.com/10.1007/s11367-016-1087-8>[Accessed 15 11 2020]; ANL GREET Model; etc.

In one embodiment, historic data 160 may be comprised of, for example, scientific journal articles including: Capper J., Is the Grass Always Greener? Comparing the Environmental Impact of Conventional, Natural and Grass-Fed Beef Production Systems, Animals, 2012; Beauchemin K. et al., Life cycle assessment of greenhouse gas emissions from beef production in western Canada: A case study, Agricultural Systems, 2010; Stanley P. et al., Impacts of soil carbon sequestration, Agricultural Systems, 2018; Machado L., Effects of marine and freshwater macroalgae on in vitro total gas and methane production, PLOS One, 2014; Pelletier N., Environmental performance in the US broiler poultry sector: Life cycle energy use and greenhouse gas, ozone depleting, acidifying and eutrophying emissions, Agricultural Systems, 2008; Verge X. et al., Greenhouse gas emissions from the Canadian pork industry, Livestock Science, 2009; Parker R. et al., Fuel use and greenhouse gas emissions of world fisheries, Nature Climate Change, 2018; etc.

In one embodiment, historic data 160 may be comprised of, for example, data from ration calculators including: Lalman D., OSU Cowculator v2.0 Beef Cow Nutrition Evaluation Software, Oklahoma State University, 2020; U of M Feedlot Ration Balancer, The University of Minnesota, 2008; Analysis & Ingredient Management, CFCTech, 2020; Swine Ration Calculator, North Carolina State University, 2000; Nates S., Aquafeed Formulation, 2015; etc.

In one embodiment, historic data 160 may be comprised of, for example, data from commercial products including: Purina 4-square Breeder Cubes, Product Label; NatureWise Meatbird 22% Crumble, Nutrena, Product Label; Producer's Pride, Hog Feed Pellets, Product Label; Bayer Environmental Science, Cimarron Max, Product Label; etc.

In one embodiment, historic data 160 may be comprised of, for example, data from industry websites including: lowcarbonranch.com, cattlefax.com, angus.org, hereford.org, drovers.com, ncba.org, nppc.org, americandairy.com, apppa.org, etc.

In one embodiment, historic data 160 may be comprised of, for example, data from experimental data including: Lopez, A., Soil Test Report, Soil No. 13983, Water, and Forage Analytical Laboratory, Oklahoma State University, 2019; Beal C., Forage Analysis Report No. 12768, Soil, Water, and Forage Analytical Laboratory, Oklahoma State University, 2019; Closeout Report, Payment Summary, Lot ID 20167210, Creekstone Farms Premium Beef LLC, Jun. 17, 2020; Robinson C., Beal Data Report OSU Feedlot Willard Sparks.xlsx, Weights and Feed Report, Oklahoma State University, 2020; Martin B., Personal Communication, Beal C., 2018; etc.

In one embodiment, the historic data 160 may be comprised of human and/or machine-readable information that may be processed, as described in more detail below, to perform additional analysis.

Model generator 122, which is described in more detail below in reference to the emissions simulator 120, may be comprised of model equations that may be derived from historic data 160. In one embodiment, the model generator 122 models the impact of various historic data 160 characteristics on emissions. This can include modeling a baseline impact on emissions for available historic data. For example, the model generator 122 may model how DMI affects emissions output by an animal. The model equations may be generated based on historic data 160 in one instance, and/or may be based on a variety of different studies and/or practical correlations that may or may not be present in the historic data 160. In one embodiment, the model equations generated by the model generator 122 may be comprised of historic data 160, on-site practices management data 130, genetic data 140, phenotypic data 145, properties data 135, that may be received from one or more other database/sources.

Properties data 135 may be comprised of physical, chemical, electrical, nuclear, magnetic, and thermal data, collectively called “property data” that can be used to, for example, determine emissions over an assessment cycle. Property data generally refers to quantifiable characteristics of a material, substance, energy form, or energy carrier, such as, for example: mass, temperature, pressure, higher heating value, lower heating value, heat of combustion, heat content, energy content, metabolizable energy, density, energy density, melting point, conductivity, resistance, heat of vaporization, current, charge, voltage, electron volts, biochemical composition, protein content, amino acid profile, fat content, lipid content, fatty acid profile, fiber content, energy content, chemical bonds, moisture content, humidity, cellulose content, carbon content, nitrogen content, radiation, conduction, convection, photons, acoustics, Reynolds number, velocity, acceleration, matter, antimatter, or other quantifiable properties. Property data for animals, crops, energy, materials or other products may be used, alone or in combination with one or more other data and/or equations described herein, to determine emissions or expected emissions over an assessment cycle.

Genetic data 140 may be comprised of genomic information about an animal or plant. In one embodiment, genetic data 140 may be comprised of, for example, data from: personal communication with animal owner, feedlot owner, animal product processing facility, meat packing facility, retail staff, etc.; NeoGen, Zoetis, SeeDNA, iQBirdTesting, Angus Genetics Inc., Method Genetics, another third party, etc.; Academic organizations such as Oklahoma State University Animal Science Department, Texas A&M AgriLife, Iowa State University Extension and Outreach, etc.; Consultants such as veterinarians, geneticists, scientists, and others skilled in the art; blockchain databases; etc. The genetic information may inform a variety of data and/or assumptions about an animal's emissions expectations. For example, certain genetic markers may be correlated to greater or lower carcass weight and/or greater or lower dry matter intake, etc.

In one embodiment, the genetic data 140 may include expected progeny performance data. Expected progeny performance data may be comprised of, for example, data from: NeoGen, Zoetis, SeeDNA, iQBirdTesting, Angus Genetics Inc., Method Genetics, another third party, etc.; Academic organizations such as Oklahoma State University Animal Science Department, Texas A&M AgriLife, Iowa State University Extension and Outreach, etc.; Consultants such as veterinarians, geneticists, scientists, and others skilled in the art; blockchain databases; etc. Specifically, expected progeny performance data for cattle may include expected progeny performance data or expected progeny differences (EPDs), e.g., expected birth weight, expected weaning weight, expected dry matter intake, expected milk production, expected mature weight, expected carcass weight, etc. Expected progeny performance data may be received from a variety of different sources and proprietary databases, or may be generated based on genotypic and/or phenotypic data. One or more model equations can be generated, identified, selected, etc., based on the expected progeny performance data.

One or more model equations can be generated, identified, selected, etc., based on genetic data.

In yet another example, phenotypic data 145 may be comprised of one or more animal's or crop's observable characteristics, including height, weight, composition, feed efficiency, etc. as described above. In one embodiment, phenotypic data 145 may be comprised of, for example, data from: personal communication with an animal or crop owner, feedlot owner or worker, feed mill owner or worker, animal product processing facility, meat packing facility, retail staff, etc. such as Abernathy Ranches, Hy-Plains Feedyard, Tyson Foods, Meyer Natural Foods LLC, Walmart, etc.; Breed association such as American Angus Association, American Hereford Association, etc.; Crop associations such as the National Corn Growers Association, etc.; Phenotypic database such as animal or crop owner records, American Angus Association's National Sire Evaluation, International Dairy Food Association, etc.; Animal management software such as CattleMax, HerdOne, Layer Farm Manager, Dairy Wellness, PigCHAMP, etc.; Crop management software; Consultants such as veterinarians, geneticists, scientists, and others skilled in the art; blockchain databases; etc. The phenotypic data 145 may inform a variety of data and/or assumptions about an animal's emissions expectations, which may be incorporated by the model generator 122. For example, certain phenotypic characteristics may be correlated to greater or lower dry matter intake, greater or lower fertilizer inputs, etc.

In one embodiment, the phenotypic data 145 may be incorporated into properties data 135. One or more model equations can be generated, identified, selected, etc., based on phenotypic data.

In another example, on-site practices management data 130 may be comprised of data obtained and/or collected from a farm. For example, in one embodiment, on-site practices management data 130 may be comprised of, for example, data from: personal communication with animal, crop, energy, material or product owner/worker, feedlot, feed mill, oil well, coal mine, material plant owner/worker, animal product processing facility, crop processing facility, energy processing facility, material processing facility, meat packing facility, retail staff, etc.; Third party certifications such as Oregon Tilth, BeefTraxx, IMI Global, etc.; Government agencies such as USDA, EPA, etc.; Academic organizations such as Oklahoma State University Animal Science Department, Texas A&M AgriLife, Iowa State University Extension and Outreach, Colorado School of Mines Energy Department, Penn State Natural Resource Department, etc.; Consultants such as veterinarians, geneticists, scientists, and others skilled in the art; blockchain databases; etc. On-site practices management data 130 may be comprised of, among other things, feeds, fertilizers, manure management, grazing management, on on-site energy use, water supply (fresh water usage), etc. Certain on-site practices management data 130 may be correlated to greater or lower greenhouse gas emissions. One or more model equations can be generated, identified, selected, etc., based on on-site practice management data.

Performance data identifier 124 identifies performance data variables from performance data 139 that may affect emissions determination. In one embodiment, identifying performance data variables may include, for example, utilizing internal protocols and training modules; Consultants such as veterinarians, geneticists, scientists, engineers, lifecycle assessment experts, and others skilled in the art. In an embodiment, utilizing one or more sources described herein, for example, expected dry matter intake and/or expected carcass weight, which may be obtained from genetic data 140, may be identified as affecting the emissions calculations—and, as such, may be identified by the performance data identifier 124. In an embodiment, utilizing one or more sources described herein, for example, crop yield and fertilizer inputs may be identified by the performance data identifier 124. In an embodiment, utilizing one or more sources described herein, for example, material yield and transport distance may be identified by the performance data identifier 124.

The emissions simulator 120 may take the data and resulting model equations into account in its model generator 122 to determine emissions emitted by one or more product system on a particular operation or collection of production facilities. In an embodiment, one or more of a plurality of approaches can be used to generate the model including, for example, practitioner formulations, regression analysis, statistical analysis, sensitivity analysis, Monte Carlo simulation, experimental trials, artificial intelligence, machine learning, training algorithms, etc.

Adjustment module 126 allows for adjustments to the model equations based on performance data variables (e.g., from identified performance data 139. That is, adjustment module 126 generates adjustment parameters accounting for differences with respect to different sets of performance data 139 and historic data 160. For example, the adjustments can be with respect to baseline emissions for a particular breed of animal or particular on-site management practices. In one exemplary embodiment, the adjustment module 126 applies scaling factors, thresholds, and/or multipliers to model equations to ensure that an appropriate emissions determination is obtained based on performance data. The amount and nature of the adjustments may be determined by the performance data and/or the data point's likely impact on the determined expected emissions values. Generally, the adjustments may be determined based on historical data 160 and/or in real time or near real time. Additionally, in another exemplary embodiment of the adjustment module 126, thresholds may be applied to the model equations (i.e., positive or negative). In one embodiment, emissions simulator 120 may generate a baseline scenario and subsequent operations might adjust model 126 components for one or more additional scenarios to compare respective emissions. In one embodiment, adjustments to model components may include, for example, utilizing expert practitioner formulations, regression analysis, statistical analysis, sensitivity analysis, Monte Carlo simulation, experimental trials, artificial intelligence, machine learning, training algorithms, etc.

Emissions calculator 128 determines the expected emissions by applying adjusted modeling equations to obtain model output. In one exemplary embodiment, emissions calculator 128 determines expected emissions by applying a simulation to create expected probability distributions. More simply, the simulation iteratively runs the emissions calculator 128 to determine the range of possible emissions and the likelihood of the actual value being within the range. One exemplary simulation to determine expected emissions values may be a Monte Carlo simulation wherein the inputs are randomized and many simulations are run in order to determine the probabilities of different outcomes. Other simulations may be used as would be apparent to one skilled in the art. In one embodiment, determining expected emissions may include, for example, utilizing lifecycle Assessment (LCA), Integrated Assessment Modeling (IAM), Environmental Impact Assessment (EIA), etc. including methods related to allocation by mass, energy, economic value, etc., system expansion, displacement credits, offsets, any number of accounting methods, any number of pre-determined frameworks and systems such as ReCiPe End Point, ReCiPe Midpoint, TRACI, SimaPro, OpenLCA, Brightway LCA, GREET, CML, eco-indicator 99, ecological footprint, EPID2003, IMPACT 2002+, USETox, Land Use Change, Indirect Land Use Change, ecosystem quality, climate change, human health, cumulative energy demand, fossil energy demand, etc.

A variety of different outputs may be determined by the emissions calculator 128, including, but not limited to values for: carbon dioxide equivalent emissions (CO₂e) absorbed on farm credit, respiratory CO₂e emissions, manure CO₂e emissions, CO₂e emissions from enteric CH₄, CO₂e emissions upstream (upstream emissions are emissions that occur outside of the production process, but are “embedded” in energy or materials that are used in the production process), CO₂e emissions from manure N₂O, CO₂e directly emitted on farm, CO₂e emissions from soil N₂O, CO₂e emissions from manure CH₄, CO₂e sequestered in soil or other media, CO₂e sequestration flows, and other greenhouse gas fluxes. In various embodiments, the units of emissions outputs may include, for example, kg CO₂e/kg carcass weight, kg CO₂e/kg live weight, kg CO₂e/animal, t CO₂e/lb meat, kg CO₂e/egg, kg CO₂e/gallon milk, kg CO₂e/kg protein, kg CO₂e/calorie, kg CO₂e/$ economic value, points of environmental impact per any given yield, moles of H+ eq, kg 2-4-d eq, kg N, kg CO₂e, kg CFC 11 eq, kg NOx eq, various combinations of units, etc. Other emissions can also be model outputs and reported in units relevant for crop production (bu, t, lb, kg, ac, ha, etc.), energy production (MJ, kWh, barrel, btu, cf, lb, kg, gal, gge, etc.), material production (t, lb, kg, m3, cf, carat, density, etc.), and others.

Network cloud 150 generally represents a network or collection of networks (such as the Internet or a corporate intranet, or a combination of both) over which the various components illustrated in FIG. 1A (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to clients' devices or other devices in response to HTTP or other requests from clients' devices or other devices. A mail server is generally capable of providing electronic mail services to various clients' devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiment, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1A, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the embodiments described herein.

FIG. 1B illustrates an example production system in accordance with various embodiments. Production system 141 can be used to represent the lifecycles (e.g., cow-calf, backgrounding, feedlot, meat packer, etc.) or parts of the lifecycles of animals being evaluated for emissions output. For example, the lifecycles can be associated with input data and output data as represented in FIG. 1B. Input 143 is associated with input parameters that impact emissions as illustrated by outputs 147. Output 147 may be comprised of greenhouse gas fluxes, product yields, or other metrics, such as one or more of the following: total lifecycle CO₂e emissions, CO₂e emissions for part of the lifecycle, CO₂e absorbed on farm credit, respiratory CO₂e emissions, manure CO₂e emissions, CO₂e emissions from enteric CH₄, CO₂e emissions upstream, CO₂e emissions from manure N₂O, CO₂e emissions direct on farm, CO₂e emissions from soil N₂O, CO₂e emissions from manure CH₄, CO₂e sequestered in soil, CO₂e credits, negative CO₂e emissions, CO₂e sequestration fluxes, carcass weight yield, by-product yields, manure yield, etc. The model may also output different combinations of emissions (e.g., emissions from feedlot only) or the model may output the total emissions for the entire pathway, total methane, and/or total N₂O, etc. As used herein, upstream emissions refer to emissions that occur outside of the beef production process, but are “embedded” in energy or materials that are used in the beef production process. Examples include nitrogen fertilizer production: ammonia is produced from natural gas and air offsite and that process causes emissions—but those emissions are attributed to the beef once the farmer purchases the nitrogen fertilizer and uses it on their farm. The same approach can be used for other materials and energy that is imported into the control volume, including, for example, feeds, fuels, seeds, etc. Although the aforementioned model outputs are detailed herein, other model outputs may be generated as would be apparent to one skilled in the art.

Model equations can characterize the production system with equations representing, for example, cow-calf models, backgrounding models, feedlot models, meat packing models, operations models, etc. The model equations can provide performance results at, for example, global, national, aggregate producer level, groups of animals, or individual animals. As will be described further in FIG. 2, a model based on the model equations can be dynamically updated based on available data. For example, adjustments can be applied to the model equations based on performance data. The adjustments can be with respect to baseline emissions as determined by, for example, inputs 143 associated with production system 141. For example, the adjustments can be with respect to baseline emissions for a particular breed of animal associated with a particular production system. In one embodiment, updating the model may include, for example, utilizing expert practitioner formulations, regression analysis, statistical analysis, sensitivity analysis, Monte Carlo simulation, experimental trials, artificial intelligence, machine learning, training algorithms, etc.

As described, a model can be dynamically updated based on available data. Example 200 of FIG. 2 illustrates an example pipeline that can be utilized in accordance with various embodiments. In this example, historic data 160 and performance data 139 are obtained and can be used to generate model 206. Model 206 can be used to determine estimated emissions for one or more animals. The model can include one or more equation components. The equation components of the model can be based on input parameters that model the impact of certain characteristics on an animal's lifecycle emissions.

For example, historic data 160 can be used to determine input parameters that impact emissions. The historic data may be compiled from academic papers, scientific literature, trade publications, etc. For example, the historic data may be comprised of information about different parts of animal lifecycle, including, for example, emissions information, economic impact information, etc. More specifically, historic data may include data grass-fed feedlot inputs including arable land data, precipitation data, atmospheric CO₂ data, etc. Performance data 139 can include, for example, expected progeny performance data, properties data, genetic data, phenotypic data, and on-site practices management data.

In this example, historic data 160 and performance data 139 are accessible to training and adjustment component 205 that includes training module 204 and adjustment module 126. Training module 204 and adjustment module 126 are illustrated within the training and adjustment component 205 for illustration purposes. They may reside inside or outside training and adjustment component 205, as would be readily understood to a person of ordinary skill in the art. Training module 204 can provide the data to model 206. In some examples, model 206 directly obtains the data and/or obtains the data via one or components and/or processes. Model 206 can include, for example, equation components based on input parameters that models the impact of certain characteristics on an animal's lifecycle emissions. For example, a model may be comprised of model equations that may be derived from historic data 160 and performance data 139.

### Model Examples

### Livestock

Models can be generated to represent the lifecycles (e.g., assessment cycles) or parts of the lifecycles (e.g., assessment cycles) of products being evaluated. This can include models for, e.g., animal products (beef, chicken, pork, milk, eggs, etc.), crops (plants, algae, fungi, cyanobacteria, bacteria, etc.), energy carriers (oil, coal, gas, solar power electricity, wind power electricity, biofuels, etc.), materials (iron, stone, silver, gold, lithium, aluminum, woods, carbon dioxide, ammonia, propylene, etc.), and other products. In accordance with various embodiments, the greenhouse gas emissions for animal products, crops, energy carriers, materials, and other products can be represented as the sum of the emissions from each part within that system. In a specific example, the greenhouse gas emissions for a livestock system can be represented as the sum of the emissions from each part within that system, which can be represented by:

GHG[CO2e]=ΣᵢⁿGHGᵢ EQ (1)

As an example, for beef production, model equations can include, for example, equations associated with greenhouse gas fluxes as follows. Photosynthesis can be represented by:

$$G H {G}_{p} [ \frac{kg {CO}_{2} e}{yr} ] = - \sum ( D M I [ \frac{k g}{yr} ] \cdot {CC}_{p} [ \frac{kg C}{kg} ] ) \cdot CF [ \frac{kg {CO}_{2}}{kg C} ] \qquad EQ ( 2 )$$

where DMI is the total dry matter intake of each material, CCₚ is the carbon content of each material, and CF is the CO₂ conversion factor (44/12). Respiration can be represented by:

$$G H {G}_{r} [ \frac{kg {CO}_{2} e}{yr} ] = D M I [ \frac{kg DM}{yr} ] \cdot RF [ \frac{kg {CO}_{2}}{kg DM} ] \qquad EQ ( 3 )$$

where RF is the respiration factor based on data from literature. CO₂ emitted from manure (GHG_{mco2}) is calculated for pasture and feedlot settings as:

$$G H {G}_{m c o 2} [ \frac{kg {CO}_{2} e}{yr} ] = VS [ \frac{kg}{yr} ] \cdot {CC}_{m} [ \frac{kg C}{k g} ] \cdot CF [ \frac{kg {CO}_{2}}{kg C} ] \qquad EQ ( 4 )$$

where CCₘ is the carbon content of the manure volatile solids (VS). Emissions from manure (GHGₘ) include several sources. Manure CH₄ emissions (GHG_{mch4}) are calculated according to the IPCC as:

$$G H {G}_{m c h 4} [ \frac{kg {CO}_{2} e}{yr} ] = VS [ \frac{kg}{yr} ] \cdot {B}_{o} [ \frac{kg {CH}_{4}}{kg} ] \cdot 0.67 \cdot MCF [ - ] \cdot {GWP}_{C {H}_{4}} [ \frac{kg {CO}_{2 e}}{kg {CH}_{4}} ] \qquad EQ ( 5 )$$

where Bₒ is the maximum CH₄ capacity, MCF is the methane conversion factor and GWP_{CH}_{₄} is the global warming potential for methane. Manure N₂O emissions (GHGₘₙ₂ₒ) are calculated as the sum of direct (GHG_{mn2oD}), indirect volatile (GHG_{mn2oV}), and indirect leaching (GHG_{mn2oL}). GHG_{mn2oD} are calculated as:

$$G H {G}_{m n 2 o D} [ \frac{kg {CO}_{2} e}{yr} ] = N [ \frac{{kg}_{N}}{yr} ] \cdot {NF}_{D} [ \frac{kg {N}_{2} O}{{kg}_{N}} ] \cdot {GWP}_{N 2 O} [ \frac{kg {CO}_{2 e}}{kg {N}_{2} O} ] \qquad EQ ( 6 )$$

where NF_{D} is the direct N₂O emissions factor (0.031 for pastures and 0.008 for feedlots), GWP_{N2O} is the N₂O global warming potential (265), and N is the total nitrogen excretion, which is calculated as:

$$N [ \frac{{kg}_{N}}{yr} ] = \sum ( D M I \cdot P C ) [ \frac{kg protein}{yr} ] \cdot \frac{N E}{N C} [ \frac{kg N}{{kg}_{p r otein}} ] \qquad EQ ( 7 )$$

where PC is the protein content of each feed, NE is the ratio of nitrogen excretion to nitrogen intake (0.93), and NC is the nitrogen conversion factor for protein (6.25). GHG_{mn2oV} are calculated as:

$$G H {G}_{mn 2 oV} [ \frac{kg {CO}_{2} e}{yr} ] = N [ \frac{{kg}_{N}}{yr} ] \cdot {NF}_{V} [ \frac{kg {N}_{2} O}{{kg}_{N}} ] \cdot {GWP}_{N 2 O} [ \frac{kg {CO}_{2 e}}{kg {N}_{2} O} ] \qquad EQ ( 8 )$$

where NF_{V} is the indirect volatile N₂O emissions factor (0.003 for pastures and 0.007 for feedlots). GHG_{mn2oL} are calculated as:

$$G H {G}_{m n 2 o L} [ \frac{kg {CO}_{2} e}{yr} ] = N [ \frac{{kg}_{N}}{yr} ] \cdot {NF}_{L} [ \frac{kg {N}_{2} O}{{kg}_{N}} ] \cdot {GWP}_{N 2 O} [ \frac{k g {CO}_{2 e}}{kg {N}_{2} O} ] \qquad EQ ( 9 )$$

where NF_{L} is the indirect leaching N₂O emissions factor (0.001 for pastures and feedlots).

Emissions from fertilizer applied to soils (GHGᵢ) are calculated using the same equations, but by replacing N with the amount of nitrogen applied as fertilizer (IN) and using the following factors: NF_{D} 0.016, NF_{V} 0.002, NF_{L} 0.004. Enteric CH₄ emissions (GHGₑ) are calculated as:

$$G H {G}_{e} [ \frac{kg {CO}_{2} e}{y r} ] = \sum ( DMI [ \frac{kg DM}{yr} ] \cdot 24.65 - 0.103 \cdot FAT ) ) [ \frac{kg {CH}_{4}}{kg DM} ] \cdot {GWP}_{C {H}_{4}} [ \frac{kg {CO}_{2 e}}{kg {CH}_{4}} ] \qquad EQ ( 10 )$$

where FAT is the amount of additional fat added to each ration. Upstream embedded GHG emissions (GHGᵤ) include the emissions generated when products brought into the control volume are produced “upstream” from the control volume system boundaries, such as purchased corn, fertilizer, fuel, etc. These emissions are calculated as,

$$G H {G}_{u} [ \frac{kg {CO}_{2} e}{yr} ] = \sum ( X [ \frac{X}{yr} ] \cdot {GHG}_{X} [ \frac{{kg}_{C O 2 e}}{X} ] ) \qquad EQ ( 11 )$$

where (X) is the amount of an imported product (e.g., corn, electricity, etc.) and GHG_{X} is the emissions impact of each product (e.g., 0.47 t CO₂e/t corn, 0.176 t CO₂e/GJ electricity, etc.) reported in a life-cycle assessment database. On-site emissions (GHGₒ) are generated from the combustion of diesel fuel and natural gas within the control volume and calculated as:

$$G H {G}_{o} [ \frac{kg {CO}_{2} e}{yr} ] = \sum ( Y [ \frac{Y}{yr} ] \cdot {GHG}_{Y} [ \frac{{kg}_{C O 2 e}}{Y} ] ) \qquad EQ ( 12 )$$

where (Y) is the amount of fuel (e.g., diesel, natural gas, etc.) and GHG_{Y} is the emissions impact of each fuel (e.g., 3.24 t CO₂e/t diesel, 0.07 t CO₂e/GJ natural gas, etc.) reported in a life-cycle assessment database. Soil carbon sequestration (or emissions) (GHG_{S}) is calculated as:

$$G H {G}_{s} [ \frac{kg {CO}_{2} e}{yr} ] = - \sum ( L [ ha ] \cdot CS [ \frac{{kg}_{C}}{h a - yr} ] ) \cdot CF [ \frac{kg {CO}_{2}}{kg C} ] \qquad EQ ( 13 )$$

where CS is the carbon sequestration (or emissions) rate per land area per year for each type of land used in the model. The total GHG emissions from the herd is calculated as:

$$GHG [ \frac{kg {CO}_{2} e}{yr} ] = G H {G}_{p} + G H {G}_{r} + G H {G}_{m} + G H {G}_{i} + G H {G}_{e} + G H {G}_{u} + G H {G}_{o} + GH {G}_{s} [ \frac{kg {CO}_{2} e}{yr} ] + G H {G}_{oth} [ \frac{kg {CO}_{2} e}{yr} ] \qquad EQ ( 14 )$$

where GHGₒₜₕ are other greenhouse gas fluxes. The GHG emissions can allocated between the carcass weight and by-products (hides, tallow, organs, etc.) based on economic allocation (below) or by different allocation methods (mass, energy, economic value, etc.) or system expansion with displacement credits. Thus, the GHG emissions per kg of carcass weight (GHG′) can be represented as:

$${GHG}^{′} [ \frac{kg {CO}_{2} e}{kg CW} ] = \frac{G H G [ \frac{kg {CO}_{2} e}{yr} ] \cdot {AF}_{CW} [ - ]}{{X}_{CW} [ \frac{kg CW}{yr} ]} \qquad EQ ( 15 )$$

where AF_{CW} is the carcass weight allocation factor and X_{CW} is the amount of carcass weight generated each year.

Model equations can characterize the production system, with, for example, equations representing cow-calf models, feeder calf models, finishing models, meat packing models, operations models, etc. The model equations can provide performance results at, for example, global, national, aggregate producer level, groups of animals, or individual animals. In an example cow-calf model, the number of calves born can be represented by:

H_{cb} [hd]=H_{c}·(1−CowDL)·(PR) [hd] EQ (16)

where H_{c} is the number of cows (e.g., 100 cows), CowDL is cow death loss rate, and PR is the pregnancy rate. The number of calves surviving to weaning (H_{w}) can be represented by:

H_{w} [hd]=H_{cb}·(1−Calf DL) [hd] EQ (17)

where CalfDL is the calf death loss rate before weaning (including birth). It is assumed that half of the calves are heifers and the other half are bulls/steers. The number of calves retained in the herd as replacement heifers (Hᵣₕ) can be represented by:

Hᵣₕ [hd]=H_{c}−(H_{c}·(1−CowDL)·(PR)) [hd] EQ (18)

The number of calves eventually slaughtered (Hₛ) can be represented by:

Hₛ [hd]=(H_{w}−H_{rg})·(1−PWDL) [hd] EQ (19)

where PWDL is the post-weaning death loss rate. The number of bulls in the herd (Hb) can be represented by:

H_{b} [hd]=H_{c}·BSR [hd] EQ (20)

where BSR is the bull stocking rate (bulls/cows) and the bull breeding life is assumed to be a predetermined year, for example, 4 years.

The amount of pastureland for the cow herd can be determined based on the dry matter intake of the cattle in the model, which can be determined for cow-calf pairs, bulls, replacement heifers, and weaned calves. Rations can be influenced by the genetic parameters in the model, as described below. Rations for cow-calf pairs can be determined for a predetermined number of time periods (e.g., four time periods: early lactation, late lactation, early gestation, and late gestation) using at least one feed ration technique known in the art. One such technique considers pasture, hay, protein supplements, and corn and a baseline average cow weight of, e.g., 1,400 lbs. Bull rations can be determined for a predetermined number of time periods (e.g., two time periods, e.g., summer and winter) consisting of pasture, hay, and protein supplements assuming a baseline average weight of, e.g., 1,600 lbs. Replacement heifers can be assumed to be fed for determined number of days, e.g., 203 days per year (e.g., November 1-April 23) with a ration of hay and protein supplements assuming a baseline average weight during feeding of, e.g., 850 lbs. The amount of land to produce forage (L_{g}) to supply the dry matter can be represented by:

$${L}_{g} = 1.25 \cdot \frac{D {M}_{g}}{P} [ \frac{lbs / yr}{\frac{lbs}{ac - yr}} ] \qquad EQ ( 21 )$$

where DM_{g} is the total annual dry matter intake of the herd for pasture and hay consumption (i.e., grass), P is the average productivity (e.g., 1.8 ton/ac-yr (USDA 2019b)), and a factor of safety (1.25) is applied. In one scenario with 100 cows, 379 ac of grassland, for example, can be used for the cow-calf segment. Mineral intake can be estimated for cattle in units of oz/hd-day, for example, depending on body weight for segments of the production pathway.

Continuing with the above example model, weaned calves can be modeled to graze on wheat pasture for a predetermined number of days (e.g., 120 days) and fed hay and protein supplements for a predetermined number of days (e.g., 41 days) for a period of time (e.g., between November 1-April 11). The amount of land for wheat pasture (L_{w}) in this example can be represented by:

$${L}_{w} [ ac ] = \frac{{H}_{b g}}{S R} [ \frac{h d}{\frac{h d}{ac}} ] \qquad EQ ( 22 )$$

where H_{bg} is the number of weaned calves backgrounded on wheat pasture and SR is the stocking rate, e.g., (0.7 hd/ac). Calves can be sent to a feedlot at a predetermined age, e.g., 13 months of age, where they can receive a feedlot ration, such as an example that consists of corn (66%), hay (16%), dried distillers grain (16%), and limestone (<1%). The average daily gain (ADG) and average carcass weight yield (CW) can be determined according to historic data 160 and performance data 139. Manure management can be modeled with a variety of systems (e.g., dry stack solid storage).

In accordance with various embodiments, the model equations can further represent on-site practice management data.

In an embodiment, one set of conditions for equation components for on-site practice management can include, for example, fertilizer being applied to the grass pasture e.g., (18 lbs DAP/ac-yr, 64 lbs ammonium nitrate/ac-yr, 41 lbs potassium chloride/ac-yr) and wheat pasture, e.g., (27 lbs DAP/ac-yr, 98 lbs ammonium nitrate/ac yr, 62 lbs potassium chloride/ac-yr). Herbicide can be modeled as applied to the grass pasture, e.g., (0.13 gal/ac-yr) and wheat pasture e.g., (0.25 gal/ac-yr), and pesticide e.g., (0.08 gal/ac-yr) and wheat seed e.g., (120 lbs/ac-yr) applied to the wheat pasture. Sub-surface water for cattle to drink can be modeled as consumed at a rate of e.g., 2.5 gal per 100 lbs of body weight for nursing cows and 1.5 gal per 100 lbs of body weight per day for all other cattle.

Continuing further, diesel fuel can be modeled as consumed for the cow-calf segment (e.g., 8 gal/cow-yr), backgrounding (e.g., 7 gal/hd-yr), and the feedlot (e.g., 20 gal/hd-yr). Diesel fuel is also consumed for transporting cattle, e.g., with fuel consumption of 0.2 gallons per mile. Electricity is consumed, e.g., for the cow-calf segment (0.5 MJ/cow-d), backgrounding (0.3 MJ/hd-d), the feedlot (0.8 MJ/hd-day), and the slaughterhouse (172 MJ/hd). Natural gas is consumed, e.g., in the feedlot (2.9 MJ/hd-d) and the slaughterhouse (681 MJ/hd).

In an embodiment, on-site management practices can include, post-weaning management, e.g., backgrounding management, direct entry to a feedlot management, grass-finished management, etc.; manure management systems, which can affect methane conversion factor (MCF), direct N2O emissions factor (NF_{D}), and indirect volatile N2O emissions factor (NF_{V}); diet formulations and feed additives that affect enteric CH4 (GHGₑ) emissions and upstream emissions (GHGᵤ); reducing or replacing inorganic fertilizer with organic fertilizer (e.g., poultry litter), which affects upstream (GHGᵤ) and direct emissions from fertilizer; replacing fossil fuel electricity with renewable electricity (e.g., solar and wind), which affects upstream emissions (GHGᵤ); replacing petroleum fuels with biofuels (e.g., biodiesel, biogas, etc.), which affects upstream (GHGᵤ) and direct emissions (GHGₒ); replacing high-GHG feedstuffs like DDGS with low-GHG feedstuffs like soymeal, which affects upstream emissions (GHG_{X}); soil carbon sequestration (GHGₛ); sequestering carbon contained in manure via thermochemical conversion or other methods; reducing the upstream emissions generated from imported products (e.g., reducing emissions from corn production or grid electricity generation), and various other management practices.

In an embodiment, direct-entry-to-feedlot management can include, for example, the situation where weaned calves are sent directly to the feedlot upon weaning and the feedlot ration consists of corn (66%), hay (16%), dried distillers grain (16%), and limestone (<1%). The average daily gain (ADG) and average carcass weight yield (CW) can be determined in accordance with any one of a number of approaches known in the art. The land for the feedlot is assumed to be negligible and manure is managed, e.g., with dry-stack solid storage. As an example, weaned calves can be modeled to enter the feedlot weighing an average of, e.g., 522 lbs and experience an average daily gain of e.g., 3.5 lbs/day for a feeding period of e.g., 233 days, yielding an average slaughter weight of e.g., 1,336 lbs, and an average carcass weight of e.g., 848 lbs.

In an embodiment, grass-finished management can include, for example, the situation where weaned calves are backgrounded on wheat or grass pasture until slaughter. The additional land for grass-finishing can be included in the calculation presented in equation 6, above. The average daily gain (ADG) and average carcass weight yield (CW) can be determined in accordance with any one of a number of approaches known in the art. In an example, grass-finishing can include, e.g., 95 ac of land grazed for 193 days with average daily gain of 1.3 lbs/hd-day, resulting in average slaughter weights of 1,136 lbs, and yielding 653 lb average carcass weights.

In an embodiment, manure management affects emissions from various production segments and for emissions from CO₂, N₂O, and CH₄. CO₂ emissions from manure represent a large carbon flux, but are typically carbon-neutral in the overall system as this carbon was initially absorbed by plants during photosynthesis (i.e., biogenic carbon). The manure emissions can be impacted by changes to the manure management system, which can alter model equations. For example, manure management systems can alter the methane conversion factor (MCF), the direct N₂O emissions factor (NF_{D}), and the indirect volatile N₂O emissions factor. For example, one scenario might have a manure management system with pasture manure left in place and dry-stack solid storage in the feedlot. Emissions for different practices (e.g., lagoons) can be calculated on a case-by-case basis by using the corresponding model parameters. In addition, a carbon sequestration credit can be obtained by collecting manure and converting it into a form for long-term storage (e.g., biochar or biogas fuel with subsequent carbon capture and storage).

### Crop

In a specific example, the greenhouse gas emissions for a crop production system can be represented as the sum of the emissions from each part within that system, which can be represented by:

GHG[CO2e]=ΣᵢⁿGHGᵢ EQ (23)

As an example, for corn production, model equations can include, for example, equations associated with greenhouse gas fluxes as follows. Photosynthesis can be represented by:

$$G H {G}_{p} [ \frac{kg {CO}_{2} e}{yr} ] = - \sum ( H [ \frac{kg}{yr} ] \cdot {CC}_{p} [ \frac{kg C}{k g} ] ) \cdot CF [ \frac{kg {CO}_{2}}{kg C} ] \qquad EQ ( 24 )$$

where H is the corn plant yield (i.e., mass of the corn plant removed from the system, including grain, stover, and other biomass), CCₚ is the carbon content of each material, and CF is the CO₂ conversion factor (44/12).

Fertilizer N₂O emissions (GHGᵢ) are calculated as the sum of direct (GHG_{mn2oD}), indirect volatile (GHG_{mn2oV}), and indirect leaching (GHG_{mn2oL}), as described in the example above.

Upstream embedded GHG emissions (GHGᵤ) include the emissions generated when products brought into the control volume are produced “upstream” from the control volume system boundaries, such as purchased fertilizer, fuel, seed, herbicide, insecticide, etc. These emissions can be represented by:

$$G H {G}_{u} [ \frac{kg {CO}_{2} e}{yr} ] = \sum ( X [ \frac{X}{yr} ] \cdot {GHG}_{X} [ \frac{{kg}_{C O 2 e}}{X} ] ) \qquad EQ ( 25 )$$

where (X) is the amount of an imported product (e.g., corn, electricity, etc.) and GHG_{X} is the emissions impact of each product (e.g., 1.462 t CO₂e/t corn seed, 0.176 t CO₂e/GJ electricity, etc.) reported in a life-cycle assessment inventory database (e.g., ecoinvent). On-site emissions (GHGₒ) are generated from the combustion of liquid fuel and natural gas within the control volume and can be represented by:

$$G H {G}_{o} [ \frac{kg {CO}_{2} e}{yr} ] = \sum ( Y [ \frac{Y}{yr} ] \cdot {GHG}_{Y} [ \frac{{kg}_{C O 2 e}}{Y} ] ) \qquad EQ ( 26 )$$

where (Y) is the amount of fuel (e.g., diesel, natural gas, etc.) and GHG_{Y} is the emissions impact of each fuel (e.g., 3.24 t CO₂e/t diesel, 0.07 t CO₂e/GJ natural gas, etc.) reported in a life-cycle assessment inventory database. Soil carbon sequestration or emissions from soils (e.g., plowing, land-use-change, etc.) (GHGₛ) can be represented by:

$$G H {G}_{s} [ \frac{kg {CO}_{2} e}{yr} ] = - \sum ( L [ ha ] \cdot CS [ \frac{{kg}_{C}}{h a - yr} ] ) \cdot CF [ \frac{kg {CO}_{2}}{kg C} ] \qquad EQ ( 27 )$$

where CS is the carbon sequestration (or emissions) rate per land area per year for each type of land used in the model. The total GHG emissions from the crop can be represented by:

$$G HG [ \frac{kg {CO}_{2} e}{yr} ] = G H {G}_{p} + G H {G}_{i} + G H {G}_{u} + G H {G}_{o} + G H {G}_{s} + G H {G}_{oth} [ \frac{kg {CO}_{2} e}{yr} ] \qquad EQ ( 28 )$$

where GHGₒₜₕ are other greenhouse gas fluxes. The GHG emissions can be allocated between the grain and by-products (e.g., stover) based on economic allocation (below) or by different allocation methods (mass, energy, etc.) or system expansion with displacement credits. Thus, the GHG emissions per kg of corn grain (GHG′) can be represented by:

$${GHG}^{′} [ \frac{kg {CO}_{2} e}{kg CW} ] = \frac{G H G [ \frac{kg {CO}_{2} e}{yr} ] \cdot {AF}_{C G} [ - ]}{{X}_{CW} [ \frac{kg CW}{y r} ]} \qquad EQ ( 29 )$$

where AF_{CG} is the grain allocation factor and X_{CG} is the amount of corn grain generated each year.

Model equations can characterize the production system, with, for example, equations representing fuel consumption for planting, spraying, harvesting, etc., electricity consumption, etc. The model equations can provide performance results at, for example, global, national, aggregate producer level, groups of crops/fields, or individual crop units. In an example corn farm model, the annual corn production can be represented by:

$$H [ t / yr ] = HY [ \frac{t}{ha - yr} ] \cdot L [ ha ] \qquad EQ ( 30 )$$

where HY is the corn yield per hectare per year and L is the land area. The amount of each input (X) (seeds, fertilizer, lime, herbicide, etc.) can be represented by:

$$X [ \frac{X}{yr} ] = {X}^{′} [ \frac{X}{ha - yr} ] \cdot L [ ha ] \qquad EQ ( 31 )$$

where X′ is the amount of each input.

Post-production processing methods to generate commodity and/or refined products from crops can also be included in the methods described here. For example, the system can include fermentation for ethanol production with distiller's grains as a co-product, corn milling for food or feed production, corn syrup production, etc. In these cases, historic data and performance data for these processes are included in the assessment.

In accordance with various embodiments, the model equations can further represent on-site practice management data.

In an embodiment, one set of conditions for equation components for on-site practice management can include, for example, fertilizer being applied to the crop e.g., (40 lbs DAP/ac-yr, 65 lbs ammonium nitrate/ac-yr, 44 lbs potassium chloride/ac-yr). Herbicide can be modeled as applied to the crop, e.g., (0.2 gal/ac-yr), pesticide e.g., (0.8 gal/ac-yr), and seed e.g., (140 lbs/ac-yr). Sub-surface water for irrigation can be modeled as consumed at a rate of e.g., 1000 gal per day.

Continuing further, diesel fuel can be modeled as consumed for the planting (e.g., 10 gal/ac), spraying (e.g., 5 gal/ac), and harvesting (e.g., 22 gal/ac). Diesel fuel is also consumed for transporting corn, e.g., with fuel consumption of 0.1 gallons per mile. Electricity is consumed, e.g., for the grain augers. Natural gas is consumed, e.g., for farm buildings.

In an embodiment, on-site management practices can include: plowing, fertilizer application methods (liquid, solid, manure, etc.), and stover collection, which impact direct N2O emissions factor (NF_{D}), and indirect volatile N2O emissions factor (NF_{V}), and soil carbon sequestration (GHGₛ); harvesting equipment, planting equipment, vehicles, etc., which can affect on-site emissions (GHGₒ); fertilizer application rates, seedings, pesticide treatments, etc., which impact upstream emissions (GHGᵤ); reducing or replacing inorganic fertilizer with organic fertilizer (e.g., poultry litter), which affects upstream (GHGᵤ) and direct emissions from fertilizer; replacing fossil fuel electricity with renewable electricity (e.g., solar and wind), which affects upstream emissions (GHGᵤ); replacing petroleum fuels with biofuels (e.g., biodiesel, biogas, etc.), which affects upstream (GHGᵤ) and direct emissions (GHGₒ); bio-based practices to enhance soil carbon sequestration (GHGₛ); sequestering carbon contained in stover via thermochemical conversion or other methods; reducing the upstream emissions generated from imported products (e.g., reducing emissions from corn seed production or grid electricity generation), and various other management practices.

In an embodiment, corn grain production can be accompanied by collection of all corn stover as a co-product, to be used for feed, fuel, fiber, or bedding.

In an embodiment, corn grain can be further process into consumer products such as corn flour, corn syrup, whole corn, etc.

In an embodiment, ethanol fermentation of corn grain can be modeled to produce ethanol, distillers' grain, carbon dioxide, and water with inputs that include sodium hydroxide, lime, urea, enzymes, acid, yeast, gasoline, water, natural gas, electricity, and other inputs. The inputs can be quantified from historic and performance data, and can be represented by:

$$X [ \frac{X}{yr} ] = {X}^{′} [ \frac{X}{kg - yr} ] \cdot Y [ kg ] \qquad EQ ( 32 )$$

where X′ is the amount of each input per kg of throughput, Y.

### Energy

In a specific example, the greenhouse gas emissions for an energy production system can be represented as the sum of the emissions from each part within that system, which can be represented by:

GHG[CO2e]=ΣᵢⁿGHGᵢ EQ (33)

As an example, for petroleum production, model equations can include, for example, equations associated with greenhouse gas fluxes from exploration (GHGₑₓ), development (GHG_{dd}), production (GHGₚₑ), waste treatment (GHG_{wt}), byproduct gas flaring (GHG_{gf}), transport (GHGₜᵣ), and refining (GHG_{ref}) (distribution and end-use can be included for “well-to-wheel” analysis) as follows. Upstream embedded GHG emissions (GHGᵤ) include the emissions generated when products brought into the control volume are produced “upstream” from the control volume system boundaries, such as chemicals, liquids, and gases (e.g., CO₂ for enhance oil recovery). These emissions are calculated as,

$$G H {G}_{u} [ \frac{kg {CO}_{2} e}{bbl} ] = \sum ( X [ \frac{X}{bbl} ] \cdot {GHG}_{X} [ \frac{{kg}_{C O 2 e}}{X} ] ) \qquad EQ ( 34 )$$

where (X) is the amount of an imported product and GHG_{X} is the emissions impact of each product (e.g., 8.1 kg CO₂e/kg CO₂, 0.176 t CO₂e/GJ electricity, etc.) reported in a life-cycle assessment inventory database (e.g., ecoinvent). On-site emissions (GHGₒ) are generated from the combustion of liquid fuel and natural gas within the control volume, as well as direct emissions of fossil molecules to the atmosphere, and be represented by:

$$G H {G}_{o} [ \frac{kg {CO}_{2} e}{bbl} ] = \sum ( Y [ \frac{Y}{bbl} ] \cdot {GHG}_{Y} [ \frac{{kg}_{C O 2 e}}{Y} ] ) \qquad EQ ( 35 )$$

where (Y) is the amount of fuel or compound (e.g., diesel, natural gas, etc.) and GHG_{Y} is the emissions impact of each fuel (e.g., 3.24 t CO₂e/t diesel burned, 0.07 t CO₂e/GJ natural gas burned, 30 kg CO₂e/kg methane released, etc.) reported in a life-cycle assessment inventory database. Each of the segments include upstream and direct sources of GHG emissions.

Emissions from exploration (GHGₑₓ) can be represented by:

$$G H {G}_{ex} [ \frac{kg {CO}_{2} e}{bbl} ] = f ( n , d , ρ , e , x ) \qquad EQ ( 36 )$$

where the emissions are a function of the number of locations explored, depth, density of soil, water, and other matter, equipment in use, and other parameters.

Emissions from drilling and development (GHG_{dd}) can be represented by:

$$G H {G}_{d d} [ \frac{kg {CO}_{2} e}{bbl} ] = f ( n , d , p , e , x ) \qquad EQ ( 37 )$$

Emissions from production and extraction (GHGₚₑ) can be represented by:

$$G H {G}_{pe} [ \frac{kg {CO}_{2} e}{bbl} ] = \sum {( G H {G}_{u} + G H {G}_{o} )}_{p e} \qquad EQ ( 38 )$$

with upstream and onsite emissions generated from materials, chemicals, liquids, gases, energy carriers, and other products utilized during production, and including fugitive emissions from the production sites, such as methane leaks.

Emissions from waste treatment (GHG_{wt}) can be represented by:

$$G H {G}_{wt} [ \frac{kg {CO}_{2} e}{bbl} ] = {( G H {G}_{u} + G H {G}_{o} )}_{wt} \qquad EQ ( 39 )$$

Emissions from byproduct gas flaring (GHG_{gf}) can be represented by:

$$G H {G}_{gf} [ \frac{kg {CO}_{2} e}{bbl} ] = {m}_{f} [ kg / bbl ] \cdot CFF [ \frac{kg {CO}_{2} e}{kg} ] \qquad EQ ( 40 )$$

where m_{f} is the mass of the gas flared and CFF is the emissions conversion factor.

Emissions from transport (GHGₜᵣ) can be represented as,

$$G H {G}_{tr} [ \frac{kg {CO}_{2} e}{bbl} ] = v [ m ] \cdot CFE [ \frac{kg CO2 e}{m} ] \qquad EQ ( 41 )$$

where v is the distance traveled and CFE is the emissions factor for the corresponding mode of transport.

Emissions from refining (GHG_{ref}) can be represented by a function:

$$G H {G}_{ref} [ \frac{kg {CO}_{2} e}{bbl} ] = f ( r , w , q , e , x ) \qquad EQ ( 42 )$$

where r is the amount of each product refined, w are the materials imported to the refinery, q are the fugitive emissions, e are the energy carries consumed, and x are other emissions.

The total GHG emissions from the energy product can be represented by:

$$GHG [ \frac{kg {CO}_{2} e}{bbl} ] = G H {G}_{e x} + G H {G}_{d d} + G H {G}_{p e} + G H {G}_{wt} + G H {G}_{gf} + G H {G}_{tr} + G H {G}_{ref} + G H {G}_{oth} [ \frac{kg {CO}_{2} e}{bbl} ] \qquad EQ ( 43 )$$

where GHGₒₜₕ are other greenhouse gas fluxes. The GHG emissions can be allocated between co-products (e.g., natural gas) based on economic allocation (below) or by different allocation methods (mass, energy, etc.) or system expansion with displacement credits. Thus, the GHG emissions per bbl of petroleum (GHG′) can be represented by:

$${GHG}^{′} [ \frac{kg {CO}_{2} e}{bbl} ] = \frac{GHG [ kg {CO}_{2} e ] \cdot {AF}_{p e t} [ - ]}{{X}_{p e t} [ bbl ]} \qquad EQ ( 44 )$$

where AFₚₑₜ is the allocation factor for petroleum and Xₚₑₜ is the amount of petroleum generated.

Model equations can characterize the production system, with, for example, equations representing fuel consumption for exploration, drilling, pumping, etc., electricity consumption, etc. The model equations can provide performance results at, for example, global, national, aggregate producer level, groups of energy carriers, or individual energy carrier units (e.g., a bbl, a t of coal, a GJ, etc.). In an example petroleum well model, the annual petroleum production can be represented by:

$$I [ bbl ] = {\int }_{i}^{t} IY [ \frac{bbl}{d} ] \cdot dt [ d ] \qquad EQ ( 45 )$$

where IY is the petroleum yield per day. The amount of each input (X) (fuel, electricity, chemicals, CO₂, etc.) can be represented by:

$$X [ X ] = {X}^{′} [ \frac{X}{d} ] \cdot t [ d ] \qquad EQ ( 46 )$$

where X′ is the amount of each input consumed per day at t is the number of days. Post-production processing methods to generate refined fuels and/or delivered consumer products from energy carriers can also be included in the methods described here. For example, the system can include hydrocracking upgrading to generate blend stocks that can be blended and transported to provide consumers with fungible fuel products (e.g., diesel). In these cases, historic data and performance data for these processes are included in the assessment.

In accordance with various embodiments, the model equations can further represent on-site practice management data, such as operating temperatures, pressures, flowrates, equipment, transport methods, etc.

In an embodiment, one set of conditions for equation components for on-site practice management can include, for example, the number of locations explored for resources (e.g., 1 or more), the depth and density of the materials explored (e.g., 4,500 m), and the energy input for the equipment used for exploration (e.g., 27 gal diesel/hour).

Continuing further, diesel fuel can be modeled as consumed for drilling operations (e.g., 117 gal per drilled foot). Electricity is consumed for the drilling process (e.g., for pumping sludge, 334 MJ/cf). Natural gas is consumed for compressors (e.g., 12,333 cf/bbl).

In an embodiment, on-site management practices can include: production flow, injection pressure, bore diameters, fugitive capture systems, operation hours depth of injection, etc., which impact energy production performance.

In an embodiment, petroleum production can be accompanied by co-production of natural gas.

In an embodiment, petroleum can be further processed into consumer products such as plastics, rubbers, detergents, etc.

In an embodiment, petroleum refining can be modeled to produce fuel oil, gasoline, diesel, jet fuel, propane, asphalt, petrochemicals, and other products with inputs that include hydrogen, hydroxide, catalyst, water, natural gas, electricity, and other inputs. The inputs can be quantified from historic and performance data, and can be represented by:

$$X [ X ] = {X}^{′} [ \frac{X}{bbl} ] \cdot Y [ bbl ] \qquad EQ ( 47 )$$

where X′ is the amount of each input per kg of throughput, Y.

### Material

In a specific example, the greenhouse gas emissions for a material production system can be represented as the sum of the emissions from each part within that system, which can be represented by:

GHG[CO2e]=ΣᵢⁿGHGᵢ EQ (48)

As an example, for iron ore production, model equations can include, for example, equations associated with greenhouse gas fluxes from exploration (GHGₑₓ), development (GHG_{dd}), production (GHGₚₑ), waste treatment (GHG_{wt}), transport (GHGₜᵣ), and refining (GHG_{ref}) (distribution and end-use can be included for “well-to-wheel” analysis) as follows. Upstream embedded GHG emissions (GHGᵤ) include the emissions generated when products brought into the control volume are produced “upstream” from the control volume system boundaries, such as chemicals, liquids, and gases. These emissions can be represented by:

$${GHG}_{u} [ \frac{kg {CO}_{2} e}{t} ] = \sum ( X [ \frac{X}{t} ] \cdot {GHG}_{X} [ \frac{{kg}_{CO 2 e}}{X} ] ) \qquad EQ ( 49 )$$

where (X) is the amount of an imported product and GHG_{X} is the emissions impact of each product (e.g., 87 g CO₂e/MJ diesel, 0.176 t CO₂e/GJ electricity, etc.) reported in a life-cycle assessment inventory database (e.g., ecoinvent). On-site emissions (GHGₒ) are generated from the combustion of liquid fuel and natural gas within the control volume, as well as direct emissions of fossil molecules to the atmosphere, and can be represented by:

$${GHG}_{o} [ \frac{kg {CO}_{2} e}{t} ] = \sum ( Y [ \frac{Y}{t} ] \cdot {GHG}_{Y} [ \frac{{kg}_{CO 2 e}}{Y} ] ) \qquad EQ ( 50 )$$

where (Y) is the amount of fuel or compound (e.g., diesel, natural gas, etc.) and GHG_{Y} is the emissions impact of each fuel (e.g., 3.24 t CO₂e/t diesel burned, 0.07 t CO₂e/GJ natural gas burned, 30 kg CO₂e/kg methane released, etc.) reported in a life-cycle assessment inventory database. Each of the segments include upstream and direct sources of GHG emissions.

Emissions from exploration (GHGₑₓ) can be represented by:

$${GHG}_{ex} [ \frac{kg {CO}_{2} e}{t} ] = f ( n , d , ρ , e , x ) \qquad EQ ( 51 )$$

where the emissions are a function of the number of locations explored, depth, density of soil, water, and other matter, equipment in use, and other parameters.

Emissions from development and mining (GHG_{dd}) can be represented by:

$${GHG}_{dd} [ \frac{kg {CO}_{2} e}{t} ] = f ( n , d , ρ , e , x ) \qquad EQ ( 52 )$$

Emissions from production and extraction (GHGₚₑ) can be represented by:

$${GHG}_{pe} [ \frac{kg {CO}_{2} e}{t} ] = \sum {( {GHG}_{u} + {GHG}_{o} )}_{pe} \qquad EQ ( 53 )$$

with upstream and onsite emissions generated from materials, chemicals, liquids, gases, energy carriers, and other products utilized during production, and including fugitive emissions from the production sites, such as methane leaks.

Emissions from waste treatment (GHG_{wt}) can be represented by:

$${GHG}_{wt} [ \frac{kg {CO}_{2} e}{t} ] = {( {GHG}_{u} + {GHG}_{o} )}_{wt} \qquad EQ ( 54 )$$

Emissions from transport (GHGₜᵣ) can be represented as,

$${GHG}_{tr} [ \frac{kg {CO}_{2} e}{t} ] = v [ m ] \cdot CFE [ \frac{kg CO 2 e}{m} ] \qquad EQ ( 55 )$$

where v is the distance traveled and CFE is the emissions factor for the corresponding mode of transport.

Emissions from final production (GHG_{ref}) can be represented by a function:

$${GHG}_{ref} [ \frac{kg {CO}_{2} e}{t} ] = f ( r , w , q , e , x ) \qquad EQ ( 56 )$$

where r is the amount of each product refined, w are the materials imported to the refinery, q are the fugitive emissions, e are the energy carries consumed, and x are other emissions.

The total GHG emissions from the material production can be represented by:

$$GHG [ \frac{kg {CO}_{2} e}{t} ] = {GHG}_{ex} + {GHG}_{dd} + {GHG}_{pe} + {GHG}_{tr} + {GHG}_{ref} + {GHG}_{oth} [ \frac{kg {CO}_{2} e}{t} ] \qquad EQ ( 57 )$$

where GHGₒₜₕ are other greenhouse gas fluxes. The GHG emissions can be allocated between co-products (e.g., waste rods) based on economic allocation (below) or by different allocation methods (mass, energy, etc.) or system expansion with displacement credits. Thus, the GHG emissions per tonne of material (GHG′) can be represented by:

$${GHG}^{′} [ \frac{kg {CO}_{2} e}{t} ] = \frac{GHG [ kg {CO}_{2} e ] \cdot {AF}_{mat} [ - ]}{{X}_{mat} [ t ]} \qquad EQ ( 58 )$$

where AFₘₐₜ is the allocation factor for the material and Xₘₐₜ is the amount of the material generated.

Model equations can characterize the production system, with, for example, equations representing fuel consumption for exploration, drilling, pumping, etc., electricity consumption, etc. The model equations can provide performance results at, for example, global, national, aggregate producer level, groups of energy carriers, or individual material units (e.g., a tonne). In an example iron ore mine model, the iron ore production can be represented by:

$$J [ t ] = {\int }_{i}^{t} JY [ \frac{t}{d} ] \cdot dt [ d ] \qquad EQ ( 59 )$$

where JY is the iron ore yield per day. The amount of each input (X) (fuel, electricity, chemicals, CO₂, etc.) can be represented by:

$$X [ X ] = {X}^{′} [ \frac{X}{d} ] \cdot t [ d ] \qquad EQ ( 60 )$$

where X′ is the amount of each input consumed per day and t is the number of days. Post-production processing methods to generate refined materials and/or delivered consumer products can also be included in the methods described here. For example, the system can include blast-furnace upgrading to generate iron. In these cases, historic data and performance data for these processes are included in the assessment.

In accordance with various embodiments, the model equations can further represent on-site practice management data, such as operating temperatures, pressures, flowrates, equipment, transport methods, etc.

In an embodiment, one set of conditions for equation components for on-site practice management can include, for example, depth of mining, equipment used, and on-site processing such as cleaning materials.

Continuing further, diesel fuel can be modeled as consumed for mining, electricity consumed pumps, and natural gas consumed for compressors.

In an embodiment, iron ore production can be accompanied by co-production of waste rods, soil, water, and other materials.

In an embodiment, iron ore can be further processed into commercial and consumer products such as steel beams.

In an embodiment, iron ore refining can be modeled to produce steel rods, and other products with inputs that include limestone, coal, metal, gases, and other inputs. The inputs can be quantified from historic and performance data, and can be represented by:

$$X [ X ] = {X}^{′} [ \frac{X}{t} ] \cdot Y [ t ] \qquad EQ ( 61 )$$

where X′ is the amount of each input per kg of throughput, Y.

### Combined Emissions Producing Systems

Examples of combined systems: In addition to the examples above, the methods described here can also apply to emission producing systems that are integrated, hybrid, synergistic, interconnected, or otherwise related. For instance, many production systems generate a combination of animal, crop, energy, material, or other products, and the modeling and assessment methods presented here apply to those systems as well. Some examples of these kinds of systems include, but are not limited to: oil and gas co-production, solar power and agricultural production (i.e., agrivoltaics), wastewater treatment integrated with algae production, integrated production of crops and livestock, hybrid systems producing dairy and beef products (i.e., “beef-on-dairy”), dairy products and biogas energy, bioenergy with carbon capture and sequestration (BECCS), algae production integrated with BECCS, oil production with lithium as a co-product, soymeal and soy-based biofuel production, corn ethanol and corn meal production, poultry and manure production for crop fertilizer and/or biofuels, macroalgae production for energy, protein, and aquaculture yields, co-grazing of multiple species, production of cotton, wheat, corn, sugar, soy, palm, potatoes, etc. for fiber, feed, and fuel, integrated agriculture of plants, animals, fungi, algae, etc., such as integrated forestry and cattle production or crop and livestock production, interconnected production of minerals on working lands, co-transportation of products, utilization of flue gases for cultivation of plants or animals, such as algae production integrated with an ammonia plant, crop rotations, hydroponic production of plants, animals, fungi, algae, etc. in a common setting, various polycultures, coffee production integrated with crops, livestock, mining, energy production, etc., indoor and vertical farming leveraging infrastructure from integrated systems, gold mining integrated with other products, such as water processing, stone production, top soil, etc., combined heat and power plants, including those integrated into other systems that use the heat and/or power, oxyfuel plants and those integrated into other processes, various chemical refining systems with multiple products, feed mills with many products, feed ration formulations with many ingredients, human food production systems, co-mining operations for multiple products, including water, salt, CO₂, lithium, coal, oil, natural gas, uranium, etc., and other processes.

Adjustment module 126 can be configured to adjust the model equations based on performance data 139, where in various embodiments the models can be adjusted in real time or near real time. For example, a decision algorithm in certain embodiments can be triggered on the input data to select appropriate equations and appropriate input variables for each scenario. In one embodiment, training module 204 can identify the data variables that may be impacted or otherwise associated with select input parameters associated with performance data 139, and can apply adjustments to model equations based on performance data 139.

In an example, the model can be adjusted to account for on-site practices management data. For example, the obtained performance data may be used as input data that may be processed to determine expected emissions in accordance with embodiments described herein. This can include, in an example, identifying on-site management practices data variables that may affect emissions calculation. For example, training module 204 can identify the data variables that may be impacted by select input parameters and can apply adjustments to model equations based on the historic data 160 and/or performance data 139. That is, adjustment parameters accounting for differences between scenarios can be generated, where the adjustments can be with respect to animal performance or particular on-site management practices. For example, industry standard conditions can be determined for a particular breed of animal and/or for particular on-site management practices. The industry standard conditions can be adjusted in accordance with embodiments described herein based on genetics and/or on-site management practices. Adjustment module 126 applies scaling factors, thresholds, and/or multipliers to model equations to ensure that an appropriate emissions determination is obtained based on on-site management data points. The amount and nature of the adjustments may be determined by the on-site management data points and/or the data point's likely impact on the determined expected emissions values.

In another example, cattle with greater reproductive efficiency, feed efficiency, and health generate fewer GHG emissions by generating more beef with fewer inputs (and thus fewer emissions). Expected progeny performance data can indicate the relative difference in performance between progeny of animals for a variety of traits, such as birth weight, dry matter intake, yearling height, carcass weight, mature weight, marbling, etc. In turn, these traits can impact the GHG emissions intensity from cattle by influencing the amounts of feeds required for the herd and the amount of manure and carcass weight generated by the herd.

In another example, model adjustments for expected progeny performance data can be based on, for example, adjustment parameters for weaning weight (WnWt), yearling weight (YrWt), calf death loss rate (CalDL), dry matter intake (DMI), average daily grain (ADG), cow death loss rate (CowDL), carcass weight (CarcWt), etc.

In yet another example, adjustment module 126 can be configured to adjust the model equations based on identified on-site management data. In one exemplary embodiment, adjustment module 126 applies scaling factors, thresholds, and/or multipliers to model equations to ensure that an appropriate emissions determination is obtained based on performance data.

In yet another example, the model can be dynamically updated based on real-time data 214. For example, sensors 219, 221, and 223 can obtain data associated with one or more animals. The sensors can include, for example, cameras, electronic scales, electronic feeders, temperature sensors, humidity sensors, movement sensors, GPS sensors, body composition sensors, health sensors, ultrasound sensors, gas sensors, feed intake measurement systems, gas sensors or calorimeter, laser-based methane sensors, soil analysis probe sensors, pH sensors, digital thermometers, digital rulers, Biolectric system sensors, ZELP sensors, Herdsy sensors, Allflex RFID sensors, GPI liquid flowmeter, GPI gas flowmeter, Milbank electricity meter, industrial controls, etc. In an embodiment, the animal performance data can include animal consumption, emissions, and behavior data. Animal consumption, emissions, and behavior data can be obtained using one or more sensors. For example, sensors can be used to monitor automatically and continuously the consumption, emissions, and the behavior of individual animals in order to predict and determine a variety of conditions relating to health, feed efficiency, animal welfare, performance, and production efficiency enabling determination of individual animal performance on different rations, response to medications, response to feed supplements, response to minerals and trace minerals, response to growth promoting substances, prediction of carcass quality, and determination of greenhouse gas and manure excretion.

It should be noted that sensors can be utilized in one or more other emissions producing systems (e.g., emissions from or otherwise caused in the production of crop, energy, material, or other product, or combination thereof) described herein. Such sensors may include, for example, a camera, a scale, a ruler, a timer, a feeder, a temperature sensor, a pressure sensor, a flow meter, an electrical sensor, a radiation sensor, a gas sensor, a liquid sensor, a humidity sensor, a movement sensor, a global positioning sensor (GPS), a soil composition sensor, a pH sensor, a body composition sensor, a health sensor, animal identification sensor, crop identification sensor, energy carrier identification sensor, material identification senso, facial identification sensor, biomedical sensor, an x-ray sensor, nuclear magnetic resonance sensor, or an ultrasound sensor, and wherein the performance data includes expected progeny performance data, expected progeny differences data, genetic data, phenotypic data, properties data and on-site practices management data associated with the selected product.

As described, adjustment module 126 can be configured to select different equations for modeling based on historic data 160 and/or performance data 139—such that the algorithm selection is triggered on the input data. The amount and nature of the adjustments may be determined by the performance data and/or the data point's likely impact on the determined expected emissions values.

For example, Table 1 illustrates a list of exemplary adjustment parameters (e.g., model adjustments) along with a range of potential model values and the example values for each parameter. The model adjustments being generated from model equations for weaning weight (WnWt), tearling weight (YrWt), calf death loss rate (CalDL), dry matter intake (DMI), average daily grain (ADG), cow death loss rate (CowDL), carcass weight (CarcWt), etc.

TABLE 1

| Adjustment | | Example | Example |
|---|---|---|---|
| Parameters | Range | Animal A | Animal B |
| Weaning Weight | 379-662 | 515 | 543 |
| (lbs) | | | |
| Yearling Weight | 630-958 | 782 | 834 |
| (lbs) | | | |
| Calf Death Loss | 1.5%-8.5% | 4.8% | 3.8% |
| Rate (CalfDL) | | | |
| Cow Death Loss | 1.5%-3.5% | 2.3% | 2.2% |
| Rate (CowDL) | | | |
| Dry Matter Intake | 0.69-1.57 | 1.02 | 0.97 |
| Factor (DMIF) | | | |
| Mature Weight | 0.91-1.12 | 1.02 | 1.05 |
| Factor (MWF) | | | |
| Milk Factor | 0.85-1.15 | 1.00 | 1.05 |
| (MILKF) | | | |
| Feedlot ADG | 3.29-4.2; (GF) | 3.5 | 3.74 |
| (lbs/d) | 1.33-1.6 | | |
| Carcass Weight | 660-993; (GF) | 843 | 879 |
| (lbs) | 483-784 | | |
| Pregnancy Rate | 0.56-1.00 | 0.87 | 0.88 |
| (PR) | | | |

Emissions component 212 can apply adjustments to the model equations based on performance data 139. Emissions component 212 is also configured to determine emissions data 222 by one or more selected animals. In an embodiment, emissions data 222 can be obtained by control component 227. Control component 227 is operable to control various appliances 225, including on-site appliances and equipment, to alter a on-site management task. Appliances 225 can include, for example, a feed formulation appliance, a manure management appliance, a gating system to control a size of a grazing area. Control component 227 can, based on emissions data 222, control appliances 225 to alter the feed formulation, grazing areas, manure management, or other on-site management task to achieve emissions goals or thresholds. Accordingly, model 206 can be generated using historic data 160, adjusted based on performance data 139, updated based on real-time data from various sensors (e.g., 219, 221, 223), and then based on emissions data 222, while control component 227 can concurrently or based on some other schedule, alter the feed formulation, grazing areas, manure management, etc. to achieve emissions thresholds. As will be described further below, emissions component 212 models the impact of available characteristics and practices on an animal's lifecycle emissions to generate emissions data.

### Emissions Simulation System

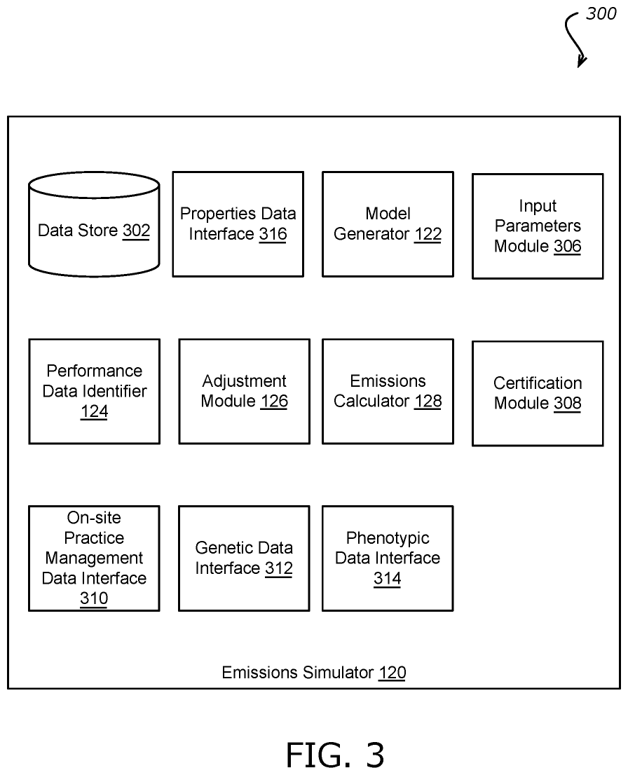

FIG. 3 below illustrates an exemplary embodiment 300 of the emissions simulator 120. As described above, the emissions simulator 120 models the impact of certain characteristics and practices on a product's lifecycle emissions.

The model output may be comprised of greenhouse gas fluxes, product yields, or other metrics, such as one or more of the following: total lifecycle CO2e emissions, CO2e emissions for part of the lifecycle, CO2e absorbed on farm credit, respiratory CO2e emissions, manure CO2e emissions, CO2e emissions from enteric CH4, CO2e emissions upstream, CO2e emissions from manure N2O, CO2e emissions direct on farm, CO2e emissions from soil N2O, CO2e emissions from manure CH4, CO2e sequestered in soil, CO2e credits, negative CO2e emissions, CO2e sequestration fluxes, carcass weight yield, by-product yields, manure yield, etc.

The model may also output different combinations of emissions (e.g., emissions from feedlot only) or the model may output the total emissions for the entire pathway, total methane, and/or total N2O, etc. The model may output different combination of emissions based on a type of data obtained, including, for example, producer-specific management practice data, performance data, energy production data, among other such data. The model can include, for example, product-centric models, animal-centric models, crop-centric models, energy production-centric models, a combination thereof, and the like. In an example, a model can be configured to quantify emissions that an animal (cow, pig, chicken, etc.) may be expected to emit over a period of time, including, for example, over the animal's lifetime. In another example, a model can be configured to quantify emissions that may be generated during the production of crops (corn, soy, wheat, algae, etc.), energy carriers, materials (e.g., plastic, iron, stone, graphite, graphene, ammonia, sulfuric acid, ethylene, propylene, lithium, silica/silicon, gold, diamonds, glass, etc.), or other products from the beginning to end of their production process. In yet another example, a model can be configured to quantify emissions that may be generated during the production of energy. In yet another example, a model can be configured to quantify emissions that may be generated during the production of material. In yet another example, a model can be configured to quantify emissions from a product. In yet another example, a model can be configured to quantify emissions from one or more (e.g., a combination of) emissions producing systems. For example, a model can be configured to quantify emissions from a crop producing system and an energy producing system, and/or segments from such systems.

In an embodiment, upstream emissions can refer to emissions that occur outside of the emissions producing process. For example, upstream emissions can refer to emissions that occur outside the beef production process, but are “embedded” in energy or materials that are used in the beef production process. Examples include nitrogen fertilizer production: ammonia is produced from natural gas and air offsite and that process causes emissions—but those emissions are attributed to the beef once the farmer purchases the nitrogen fertilizer and uses it on their farm. The same approach can be used for other materials and energy that is imported into the control volume, including, for example, feeds, fuels, seeds, etc.

Although the aforementioned model outputs are detailed herein, other model outputs may be generated as would be apparent to one skilled in the art. The emissions simulator 120 includes data store 302, model generator 122, input parameters module 306, performance data identifier 124, adjustment module 126, emissions calculator 128, certification module 308 and on-site practice management data interface 310, genetic data interface 312, phenotypic data interface 314, and properties data interface 316. The emissions simulator 120 may also include a control volume analysis. Other generators, parameters, modules and interfaces may be used, as would be readily understood by a person of ordinary skill in the art, without departing from the scope of the embodiments described herein.

The data store 302 is illustrated within the emissions simulator 120 for illustration purposes. It may reside inside or outside the emissions simulator 120, as would be readily understood to a person of ordinary skill in the art. Exemplary data stores 302 include a database for storing data, a database for storing input parameters, a database for storing calculated emissions, a database for storing models. Other databases may be used, as would be readily understood to a person of ordinary skill in the art, without departing from the scope of the embodiments described herein.

The input parameters module 306 utilizes input parameters from selected data, including, for example, producer-specific management practice data, performance data, energy production data, among other such data and other sources to create a model for the input parameters. In an example, one or more input parameters can be added or removed or otherwise selected based on the presence (or absence) of historic emissions and expected emissions data and performance data including expected progeny performance data, on-site practices management data, genetic data, phenotypic data, properties data.

A database server or other appropriate component is generally capable of providing an interface for managing data stored in one or more data stores. For example, on-site practice management data interface 310 communicates with farms and/or other relevant databases to obtain on-site practice management data. On-site practice management data may be used as input parameters in the adjusted model equations to update expected emissions calculations. On-site practice management data may be comprised of one or more of the following: feeds, fertilizers, manure management data, grazing management data, on-farm energy use data, and water supply/fresh water usage data. Although farms are described herein, ranches, factories, or other locations may interface with embodiments described herein as would be apparent to one skilled in the art.

Genetic data interface 312 interfaces with the genetic data 140 databases to import data and apply appropriate scaling, thresholding and other calculations that may be relevant or appropriate to incorporate this data into the model generator 122.

The phenotypic data 145 interfaces with the phenotypic data 145 databases to import data and apply appropriate scaling, thresholding, and other calculations that may be relevant or appropriate to incorporate this data into the model generator 122.

Properties data interface 316 interfaces with properties data 135 databases to import data and apply appropriate scaling, thresholding and other calculations that may be relevant or appropriate to incorporate this data into the model generator 122.

Model generator 122, performance data identifier 124, adjustment module 126, and emissions calculator 128, are described above in reference to FIG. 1A.

Certification module 308 assigns one or more certification(s) if the expected emissions are calculated to be above, below, or otherwise satisfy a designated threshold. In one embodiment, the certification module 308 indicates the amount of greenhouse gas emissions that an emissions producing system (e.g., emissions from an animal, crop, energy, material or other product) has emitted and/or expected to emit in accordance with the calculation system described herein. Certifications could include transaction related to product labeling, emissions limits (caps), emissions trades, emissions taxes, emissions offset (i.e., credits), other emissions transactions, etc.

### Processes for Using Performance Data to Make Estimates

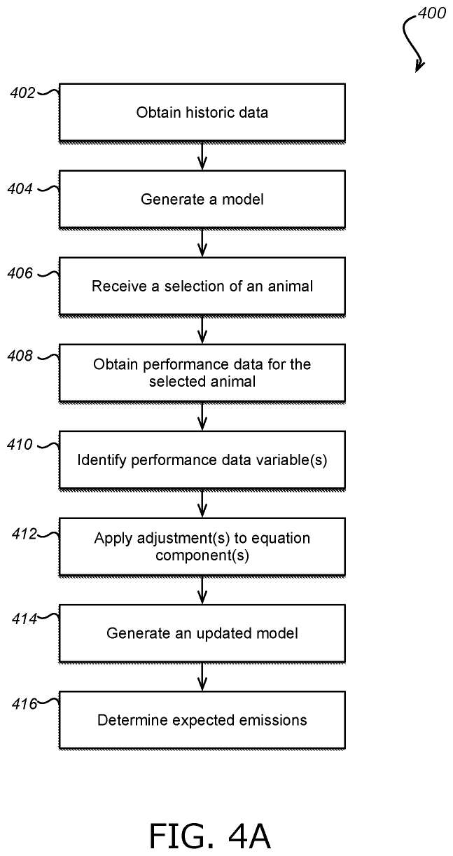

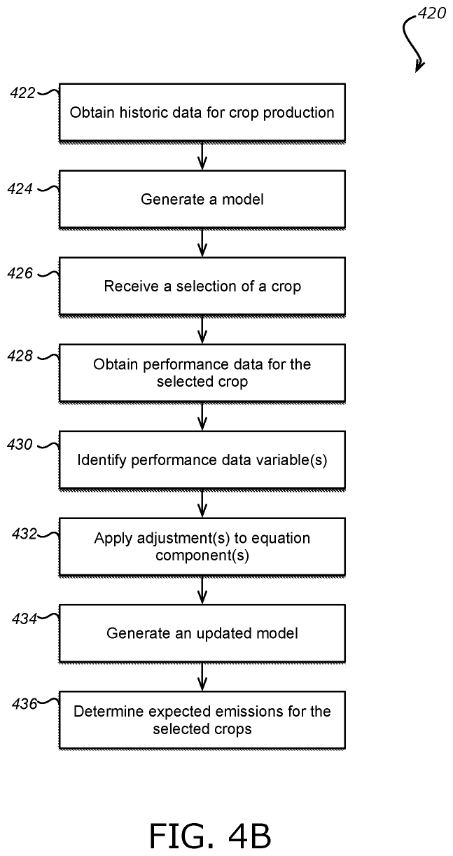

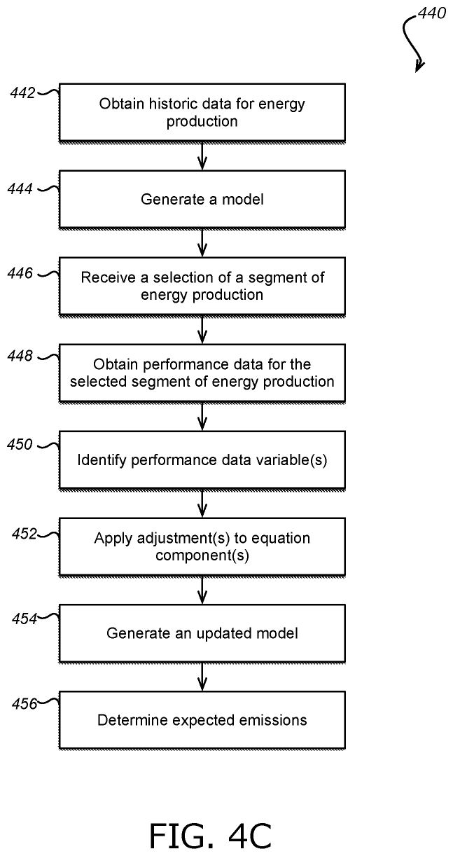

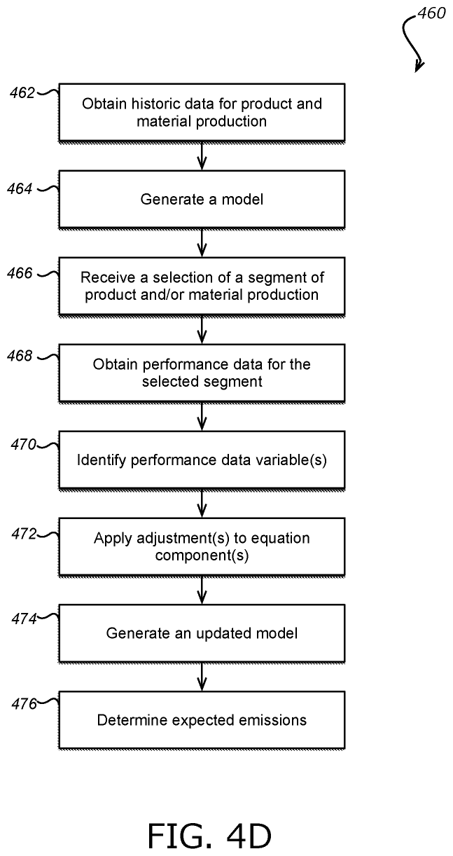

FIG. 4A below illustrates an exemplary process 400 for estimating emissions for selected emissions producing systems (e.g., emissions from an animal, crop, energy, material, or other product, or combination thereof) in accordance with an exemplary embodiment. It should be understood that, for any process discussed herein, there can be additional, fewer, or alternative steps, performed in similar or different orders, or in parallel, within the scope of the various embodiments unless otherwise stated.

In this example, the process starts by obtaining 402 historic data from a variety of sources such as academic papers, scientific literature, trade publications, experimental data, etc. In one embodiment, the relevant papers may specifically study the effects of various input parameters on emissions. In the same or other embodiments, the relevant papers may study product characteristics data that may or may not specifically analyze or study animal characteristic data and its direct impact on emissions. For example, the historic data may be comprised of information about different parts of animal lifecycle, including, but not limited to emissions information, economic impact information, etc. In various embodiments, the historic data may be comprised of information operable to quantify emissions from production of animal, crop, energy, material, and other products (herein also referred to as “emissions producing systems”). In one embodiment, the historic data may be associated with greenhouse gas emissions that an animal may be expected to emit over a period of time, including, for example, over the animal's lifetime. For example, the data may be compiled from academic papers, scientific literature, trade publications, experimental data, etc. In one embodiment, the historic data may be comprised of, for example, dry matter intake (DMI) and its effect on emissions results. In one embodiment, the historic data may be comprised of human and/or machine-readable information that may be processed, as described in more detail below, to perform additional analysis.

The data can be associated with a plurality of input parameters, where individual input parameters may be associated with equation components that cause a threshold level of change in emissions for at least one emissions producing system. For example, the relevant papers may specifically study the effects of various input parameters on emissions. This can include, for example, the presentation of one or more equation components modeling the effects of various input parameters on emission. Example input parameters include emissions from, e.g., cow-calf, backgrounding, feedlot, meat packing segments, production of crops segments (e.g., farms, elevators, mills, food processing facilities, etc.), energy producing processes (e.g., wells, mines, refineries, etc.), material processes (e.g., wells, mines, ships, trucks, refineries, chemical processing plants, etc.), or other products from the beginning to end of their production process, and provide a measure of lifecycle emissions.

The input parameters can be associated with equation components. An equation component can model the impact of certain input parameters on emissions for a variety production systems for animal, crop, energy, material, and other products. That is, an equation component can be configured to quantify an amount of emissions based on particular data. For example, electricity and natural gas consumed in the meat packing segment can be represented by at least one equation component. In this example, the equation component can model the impact of electricity and natural gas consumed to the life-cycle emissions of a beef carcass.

The process obtains or identifies one or more equation components to generate 404 a model based on the historic data and/or other data such as performance data including expected progeny performance data, properties data, genetic data, phenotypic data, and/or on-site practices management data. The generated model may incapsulate the relationship between input parameters, other data points, and their likely impact on greenhouse gas emissions. The model, including the equation components, can quantify an amount of emissions by animals, crop, energy, material, and other product production systems. In the situation where the emissions producing system includes animals, the model can quantify the amount of emissions by a group of animals based on available data. The model equations for the model can include, for example, equations representing cow-calf models, feeder calf models, finishing models, meat packing models, operations models, etc. In a specific example, an equation component of the model may quantify how DMI affects emissions output by an animal. The model may include equation components based on historic data in one instance, and/or may be based on a variety of different studies and/or practical correlations that may or may not be present in the historic data. In one embodiment, the equation components may be comprised of historic data, on-site practices and protocol data, genetic data, phenotypic data, etc. that may be received from one or more other database/sources.

Continuing with the example of an animal-based emissions producing system, a selection of an animal or a group of animals can be received 406. For example, an animal or a group of animals can be associated with a unique identifier (e.g., number, name, etc.). The identifier can comprise, for example, a tag, tattoo, metal ID clip, RFID button, freeze brand, hot brand, microchip, animal recognition technology, DNA testing, pedigree registration, etc. Similar selections can be made for crops, energy, material, and other products using identifiers.

In this example, the identifier can be associated with data corresponding to the selected animal or group of animals. The data can include, for example, animal characteristic data, performance data, and the like. Performance data can be associated with expected progeny performance data, on-site management practices, properties data, genetic data, phenotypic data, etc. Some animals can be identified by numbers, letters, shapes, codes, images, RFID data, bar codes, scannable codes, brands, blockchain data, etc. Crop, energy, material, and other products can be identified with numbers, letters, shapes, codes, images, RFID tags, bar codes, scannable codes, brands, blockchain data, etc.

Based on the selected animal(s), performance data can be obtained 408, which, as described above, may be comprised of expected progeny performance data, genetic data, phenotypic data, and/or on-site practices management data. In an example, performance data associated with on-site practices management can include, for example, manure management, grazing management, on on-site energy use, water supply (fresh water usage), etc. Performance data associated with genetic characteristics include, for example, weaning weight, yearling weight, calf death loss rate, cow death loss rate, mature weight factor, milk factor, feedlot ADG, carcass weight, pregnancy rate. At least a portion of the data may be obtained in real-time. For example, animal consumption, emissions, and behavior data can be obtained using one or more sensors positioned in accordance with different parts of animal lifecycle. For example, sensors can be used to monitor automatically and continuously the consumption, emissions, and the behavior of individual animals. The sensors can include, for example, cameras, electronic scales, electronic feeders, RFID, temperature sensors, gas sensors, etc.

In an embodiment, the obtained performance data may be used to update the generated model. For example, the obtained performance data may be used as input data that may be processed to determine expected emissions in accordance with embodiments described herein. This can include, for example, identifying 410 performance data variables that may affect emissions calculation. For example, as described, an equation component can model the impact of certain input parameters on an animal's lifecycle emissions. In this situation, an equation component can be configured to quantify an amount of emissions based on certain performance data, such as on-site management practice data. For example, the impact of fat into feedlot diets, converting feedlot manure management to daily manure spread rather than solid storage, the use of inorganic fertilizers, replacing all diesel with biodiesel, replacing all electricity with solar power, soil carbon sequestration, emissions-reducing feed additives, etc. can be represented by at least one equation component.

Identifying a data variable associated with at least one equation component of the plurality of equation components can include, for example, identifying data variables associated with equation components determined to impact or otherwise change a level of emissions at least a threshold amount. For example, as described, an equation component can model the impact of certain input parameters on an animal's lifecycle emissions. As such, an equation component can be configured to quantify an amount of emissions based on certain performance data, such as phenotype data. In a specific example, an equation component can be configured to quantify an amount of emissions based on reproductive efficiency and feed efficiency. Continuing with this example, with respect to reproductive efficiency, higher death loss leads to more GHG emissions per kilogram of beef produced by the herd because there is a lower beef yield due to the death loss. Such a relationship can be represented by at least one equation component. Further, a data variable associated with the relationship can be identified.

In yet another example, the following relationships can be represented by at least one equation component, where a data variable associated with the relationship can be identified: the pregnancy rate of a herd. Open cows (non-pregnant) produce a lot of emissions and no beef, so reproductive efficiency is an important factor in minimizing GHG emissions of a herd.

With respect to feed efficiency, weaning weight (WW) can be associated with an equation component as a direct impact on the modeled weaning weight of calves. For example, a higher weaning weight results in less time and feed requirements (and therefore lower GHG emissions) to bring an animal from weaning to market conditions (slaughter). Yearling Weight (YW) can be associated with an equation component to quantify the impact on the yearling weight of calves and time-to-slaughter, feed inputs, and GHG emissions in the model. Carcass weight (CW) measures the impacts of carcass yield, where a higher CW directly equates to a greater quantity of beef produced, and thus a lower emissions intensity per lb of beef. Average daily gain (ADG) and dry matter intake (DMI) influence feed efficiency and can be associated with an equation component to quantify the impact on the slaughter calves throughout their life. Mature weight (MW) can be associated with an equation component to quantify the impact of feed intake in the model as larger cows require more feed.

In another example, as described, an equation component can model the impact of certain input parameters on an animal's lifecycle emissions. As such, an equation component can be configured to quantify an amount of emissions based on certain performance data, such as genetic data. In a specific example, an equation component can be configured to quantify an amount of emissions based on reproductive efficiency and feed efficiency. Continuing with this example, with respect to reproductive efficiency, assume calving ease direct (CED) refers to the likelihood that a heifer that has been serviced by a sire will successfully deliver a live calf (the calf being the sire's progeny). In this example, a low calving ease expected progeny performance data may result in a higher death loss, leading to more GHG emissions per kilogram of beef produced by the herd because there is a lower beef yield due to the death loss. Such a relationship can be represented by at least one equation component. Further, a data variable associated with the relationship can be identified.

In another example, calving ease maternal (CEM) is similar to CED, but refers to the calving ease for that sire's daughter (when his daughter has a calf of her own). Such a relationship can be represented by at least one equation component. Further, a data variable associated with the relationship can be identified.

In yet another example, birth weight (BW) can similarly impact live calf birth rate as larger calves are more difficult to birth. Such a relationship can be represented by at least one equation component. Further, a data variable associated with the relationship can be identified.

In yet another example, in the situation that a certain group of animals has better CED, BW, and CEM performance than another group, the result can be lower calf death loss rate and lower cow death loss rate. Such relationships can be represented by at least one equation component. Further, a data variable associated with the relationship can be identified.

In other examples, the following relationships can be represented by at least one equation component, where a data variable associated with the relationship can be identified: the pregnancy rate of a herd given, for example, given heifer pregnancy (HP) as a measure of pregnancy rate in a sire's daughters; scrotal circumference (SC) of a sire impacts conception rates of a sire's sons, but has also has been correlated to reproductive efficiency of his daughters. SC can be considered a threshold trait, meaning there is no impact of SC on herd performance unless SC is abnormally low. Open cows (non-pregnant) produce a lot of emissions and no beef, so reproductive efficiency is an important factor in minimizing GHG emissions of a herd. For example, an animal or group of animals might have a higher SC than another group and a slightly higher HP than the baseline, resulting in a slightly higher predicted pregnancy rate.

With respect to feed efficiency, weaning weight (WW) EPD can be associated with an equation component as a direct impact on the modeled weaning weight of calves. For example, a higher weaning weight results in less time and feed requirements (and therefore lower GHG emissions) to bring an animal from weaning to market conditions (slaughter). Yearling Weight (YW) can be associated with an equation component to quantify the impact on the yearling weight of calves and time-to-slaughter, feed inputs, and GHG emissions in the model. Carcass weight (CW) measures the impacts of carcass yield, where a higher CW directly equates to a greater quantity of beef produced, and thus a lower emissions intensity per lb of beef. Residual average daily gain (RADG) and dry matter intake (DMI) influence feed efficiency and can be associated with an equation component to quantify the impact on the slaughter calves throughout their life, and also on replacement females throughout their breeding life. Milk (MILK) can be associated with an equation component to quantify the impact of the feed intake of cows during lactation periods, where a higher MILK EPD can cause an increased feed intake requirement. Mature weight (MW) can be associated with an equation component to quantify the impact of feed intake in the model as larger cows require more feed.

In another example, expected dry matter intake and/or expected carcass weight, which may ultimately affect the emissions calculations—and, as such, may be identified by the process. As an example, expected progeny performance data can be obtained from breed organizations, such as the American Angus Association, American Herford Association, etc.

The process applies 412 adjustments to identified data variables of equation components to generate 414 an updated model based on identified performance data points, the updated model quantifying an amount of emissions by the selected animal during a lifetime of the animal (or production process of crops, energy, material, or other products).

In one exemplary embodiment, the process applies scaling factors, thresholds, and/or multipliers to model equation components to ensure that an appropriate emissions calculation is obtained based on performance data. The amount and nature of the adjustments may be calculated by the performance data and/or the data point's likely impact on the calculated expected emissions values. Generally, the adjustments may be calculated based on historical performance data and/or in real time or near real time. For example, adjustment parameters can be determined along with a range of potential model values and the baseline value for each parameter. As described, adjustment parameters can include weaning weight (WnWt), yearling weight (YrWt), calf death loss rate (CalDL), dry matter intake (DMI), average daily grain (ADG), cow death loss rate (CowDL), carcass weight (CarcWt), etc. Additionally, in another exemplary embodiment of the adjustment module 126, thresholds may be applied to the model equations (i.e., positive or negative).

It should be noted that approaches described herein can be utilized to optimize other types of models including, for example, product-centric models, crop-centric models, energy production-centric models, and the like.

The process determines 416 expected emissions by applying adjusted modeling equations to obtain model output. In one exemplary embodiment, the process determines expected emissions by applying a simulation to create expected probability distributions. In one embodiment, the process determines or models the results many times to figure out the range of possible emissions and the likelihood of the actual value being within the range. One exemplary simulation to calculate expected emissions values may be a Monte Carlo simulation wherein the input is random and many simulations are run in order to determine the probabilities of different outcomes. Other simulations may be used as would be apparent to one skilled in the art.

A variety of different outputs may be calculated, including, but not limited to values for greenhouse gas fluxes, product yields, or other metrics, such as: total lifecycle CO₂e emissions, CO₂e emissions for part of the lifecycle, CO₂e absorbed on farm credit, respiratory CO₂e emissions, manure CO₂e emissions, CO₂e emissions from enteric CH₄, CO₂e emissions upstream (upstream emissions are emissions that occur outside of the production process, but are “embedded” in energy or materials that are used in the production process), CO₂e emissions from manure N₂O, CO₂e directly emitted on farm, CO₂e emissions from soil N₂O, CO₂e emissions from soil N₂O, CO₂e emissions from soil N₂O, CO₂e emissions from manure CH₄, CO₂e sequestered in soil or other media, CO₂e credits, negative CO₂e emissions, CO₂e sequestration fluxes, carcass weight yield, by-product yields, manure yield, etc. Output metrics can include a variety of measures, such as, but not limited to: kg CO₂e/kg carcass weight, kg CO₂e/head, etc.

FIG. 4B below illustrates an exemplary process 420 for estimating emissions for the production of selected crops in accordance with an exemplary embodiment. In this example, the process starts by obtaining 422 historic data from a variety of sources such as academic papers, scientific literature, trade publications, experimental data, etc. In one embodiment, the relevant papers may specifically study the effects of various input parameters on emissions. In the same or other embodiments, the relevant papers may study product characteristics data that may or may not specifically analyze or study crop production characteristic data and its direct impact on emissions. For example, the historic data may be comprised of, for example, information about different parts of crop production system, including, but not limited to performance data, emissions information, economic impact information, etc. Specifically, historic data for crops may include crop yield relationships with fertilizer, geographic production data, genetic impacts, pest management data, water requirements, etc. In one embodiment, the historic data may be associated with greenhouse gas emissions that a crop may be expected to emit over a period of time, including, for example, planting, harvesting, transporting, or a segment thereof. For example, the data may be compiled from academic papers, scientific literature, trade publications, experimental data, etc. In one embodiment, the historic data may be comprised of, for example, equipment use, energy use, or combination thereof and its effect on emissions results. In one embodiment, the historic data may be comprised of human and/or machine-readable information that may be processed, as described in more detail below, to perform additional analysis.

The data can be associated with a plurality of input parameters, where individual input parameters may be associated with equation components that cause a threshold level of change in emissions for at least one emissions producing system. For example, the relevant papers may specifically study the effects of various input parameters on emissions. This can include, for example, the presentation of one or more equation components modeling the effects of various input parameters on emission. Example input parameters include emissions from, e.g., crop-type, soil, equipment, crop harvesting segments, production of crops segments (e.g., farms, elevators, mills, food processing facilities, etc.), from the beginning to end of their production process, and provide a measure of lifecycle emissions.

The input parameters can be associated with equation components. An equation component can model the impact of certain input parameters on emissions for a variety production systems for crop. That is, an equation component can be configured to quantify an amount of emissions based on particular data. For example, crop type can be represented by at least one equation component. In this example, the equation component can model the impact of the type of crop on the life-cycle emissions of the crop. In another example, each of one or more crop segments (e.g., farms, elevators, mills, food processing facilities, etc.) can be represented by at least one equation component. In this example, the equation components can model the impact of the crop segment on the life-cycle emissions of the crop.

The process obtains or identifies one or more equation components to generate 424 a model based on the historic data and/or other data such as performance data including expected progeny performance data, properties data, genetic data, phenotypic data, and/or on-site practices management data. The generated model may incapsulate the relationship between input parameters, other data points, and their likely impact on greenhouse gas emissions.

The model, including the equation components, can quantify an amount of emissions by crop production systems. In an example, the model can quantify the amount of emissions for the production of a group of crops based on available data. The model equations for the model can include, for example, equations representing crop segment models such as, for example, farm models, elevator modes, mill models, food processing facility models, food transportation models, operations models, etc. In a specific example, an equation component of the model may quantify how a food processing facility affects emissions output by a crop. The model may include equation components based on historic data in one instance, and/or may be based on a variety of different studies and/or practical correlations that may or may not be present in the historic data. In one embodiment, the equation components may be comprised of historic data, on-site practices and protocol data that may be received from one or more other database/sources.

In an embodiment, the model may include equation components based on data obtained using one or more sensors. For example, sensors can be used to monitor automatically and continuously the growth, flux, and ecosystem impacts of crops. The data can be used to predict and determine a variety of conditions relating to health, performance, and production efficiency enabling determination of specific performance in response to minerals and trace minerals, growth promoting substances, fertilization, photosynthesis, evapotranspiration, growth rate, composition, gas flux, liquid flows.

A selection of a crop can be received 426. For example, crop or section of a crop can be associated with a unique identifier (e.g., number, name, etc.). The identifier can comprise, for example, a tag, RFID button, crop recognition technology, DNA testing, etc. In this example, the identifier can be associated with data corresponding to the selected crop. The data can include, for example, crop characteristic data, performance data, and the like. Performance data can be associated with on-site management practices, properties data, genetic data, phenotypic data, etc. Some crops can be identified with numbers, letters, shapes, codes, images, RFID tags, bar codes, scannable codes, brands, blockchain data, etc.

Based on the selected crop or group of crops, performance data can be obtained 428, which, as described above, may be comprised of expected progeny performance data, genetic data, phenotypic data, and/or on-site practices management data. In an example, the obtained performance data may be used to update the generated model. For example, the obtained performance data may be used as input data that may be processed to determine expected emissions in accordance with embodiments described herein. This can include, for example, identifying 430 performance data variables that may affect emissions calculation. For example, as described, an equation component can model the impact of certain input parameters on a crop's lifecycle emissions. In this situation, an equation component can be configured to quantify an amount of emissions based on certain performance data, such as on-site management practice data. For example, the impact of fertilizer application rate, electricity source, seeding rate, seed type, irrigation, herbicide application, pesticide application, harvesting equipment, harvesting fuel, transport equipment, transport fuel, etc. can be used.

Identifying a data variable associated with at least one equation component of the plurality of equation components can include, for example, identifying data variables associated with equation components determined to impact or otherwise change a level of emissions at least a threshold amount. For example, as described, an equation component can model the impact of certain input parameters on a crop's lifecycle emissions. As such, an equation component can be configured to quantify an amount of emissions based on certain performance data. In a specific example, an equation component can be configured to quantify an amount of emissions based on a crop yield relationship with fertilizer. Such a relationship can be represented by at least one equation component. Further, a data variable associated with the relationship can be identified.

The process applies 432 adjustments to identified data variables of equation components to generate 434 an updated model based on identified performance data points, the updated model quantifying an amount of emissions for the production of the selected crop during a selected segment (including, e.g., the entire production process) of the production process of the crop. In one exemplary embodiment, the process applies scaling factors, thresholds, and/or multipliers to model equation components to ensure that an appropriate emissions calculation is obtained based on performance data. The amount and nature of the adjustments may be calculated by the performance data and/or the data point's likely impact on the calculated expected emissions values. Generally, the adjustments may be calculated based on historical performance data and/or in real time or near real time. For example, adjustment parameters can be determined along with a range of potential model values and the baseline value for each parameter. Additionally, in another exemplary embodiment of the adjustment module 126, thresholds may be applied to the model equations (i.e., positive or negative).

The process determines 436 expected emissions by applying adjusted modeling equations to obtain model output. In one exemplary embodiment, the process determines expected emissions by applying a simulation to create expected probability distributions. In one embodiment, the process determines or models the results many times to figure out the range of possible emissions and the likelihood of the actual value being within the range. One exemplary simulation to calculate expected emissions values may be a Monte Carlo simulation wherein the input is random and many simulations are run in order to determine the probabilities of different outcomes. Other simulations may be used as would be apparent to one skilled in the art.

A variety of different outputs may be calculated, including, but not limited to values for greenhouse gas fluxes, product yields, or other metrics, such as: total lifecycle CO₂e emissions, CO₂e emissions for part of the lifecycle, CO₂e absorbed on farm credit, respiratory CO₂e emissions, manure CO₂e emissions, CO₂e emissions from enteric CH₄, CO₂e emissions upstream (upstream emissions are emissions that occur outside of the production process, but are “embedded” in energy or materials that are used in the production process), CO₂e emissions from manure N₂O, CO₂e directly emitted on farm, CO₂e emissions from soil N₂O, CO₂e emissions from soil N₂O, CO₂e emissions from soil N₂O, CO₂e emissions from manure CH₄, CO₂e sequestered in soil or other media, CO₂e credits, negative CO₂e emissions, CO₂e sequestration fluxes, carcass weight yield, by-product yields, manure yield, etc. Output metrics can include a variety of measures, such as, but not limited to: kg CO₂e/kg carcass weight, kg CO₂e/head, etc. In various embodiments, the units of emissions outputs may include, for example, bu, t, lb, kg, ac, ha, etc.

FIG. 4C below illustrates an exemplary process 440 for estimating emissions for the production of energy in accordance with an exemplary embodiment. In this example, the process starts by obtaining 442 historic data from a variety of sources such as academic papers, scientific literature, trade publications, experimental data, etc. In one embodiment, the relevant papers may specifically study the effects of various input parameters on emissions. In the same or other embodiments, the relevant papers may study product characteristics data that may or may not specifically analyze or study energy production characteristic data and its direct impact on emissions. For example, the historic data may be comprised of, for example, information about different parts of energy production systems, including, but not limited to performance data, emissions information, economic impact information, etc. Specifically, historic data for energy may include production yields, energy input requirements, transport requirements, end-use combustion performance, etc. In one embodiment, the historic data may be comprised of human and/or machine-readable information that may be processed, as described in more detail below, to perform additional analysis.

The data can be associated with a plurality of input parameters, where individual input parameters may be associated with equation components that cause a threshold level of change in emissions for at least one emissions producing system or segment thereof. For example, the relevant papers may specifically study the effects of various input parameters on emissions. This can include, for example, the presentation of one or more equation components modeling the effects of various input parameters on emission.

Example input parameters include emissions derived from, e.g., property data. Property data includes physical, chemical, electrical, nuclear, magnetic, and thermal data Property data generally refers to quantifiable characteristics of a material, substance, energy form, or energy carrier, such as, for example: mass, temperature, pressure, higher heating value, lower heating value, heat of combustion, heat content, energy content, metabolizable energy, density, energy density, melting point, conductivity, resistance, heat of vaporization, current, charge, voltage, electron volts, biochemical composition, protein content, amino acid profile, fat content, lipid content, fatty acid profile, fiber content, energy content, chemical bonds, moisture content, humidity, cellulose content, carbon content, nitrogen content, radiation, conduction, convection, photons, acoustics, Reynolds number, velocity, acceleration, matter, antimatter, or other quantifiable properties.

Additional data may be obtained from on-site management and operations of oil-and-gas wells, coal mines, photovoltaic systems, wind power systems, refineries, biorefineries, transport systems, distribution systems, or other site-specific operations within a production system. For example, on-site data might be collected for methane leaks from a natural gas operation or carbon dioxide flue gas from a biorefinery. Similar data may be obtained from on-site management and operations of gold mines, lithium recovery ponds, direct air capture machines producing CO2 or other gases, bentonite mines, propylene chemical plants, sodium hydroxide chemical plants, or other site-specific operations within a production system. For example, on-site data might be collected for energy consumption, GHG emissions from, and CO2 collection by a direct air capture CO2 plant.

The input parameters can be associated with equation components. An equation component can model the impact of certain input parameters on emissions for a variety production systems for energy. That is, an equation component can be configured to quantify an amount of emissions based on particular data. For example, energy transportation can be represented by at least one equation component. In this example, the equation component can model the impact of transporting the energy on the life-cycle emissions for energy production. In another example, each of one or more energy production segments can be represented by at least one equation component. In this example, the equation components can model the impact of producing the energy on the life-cycle emissions of the energy system. In another example, production for storing energy can be represented by at least one equation component.

The process obtains or identifies one or more equation components to generate 444 a model based on the historic data and/or other data such as performance data. The generated model may incapsulate the relationship between input parameters, other data points, and their likely impact on greenhouse gas emissions. The model, including the equation components, can quantify an amount of emissions by energy production systems. The model equations for the model can include, for example, equations representing energy production segment models such as, for example, production yield models, energy transportation models, operations models, etc. In a specific example, an equation component of the model may quantify how production for energy storage and/or transportation affects emissions output by an energy production system. The model may include equation components based on historic data in one instance, and/or may be based on a variety of different studies and/or practical correlations that may or may not be present in the historic data. In one embodiment, the equation components may be comprised of historic data, on-site practices and protocol data that may be received from one or more other database/sources.

Continuing with the example of an energy-based emissions producing system, a selection of a segment of energy production can be received 446. The segment (e.g., site location build, energy harvesting, energy storage, energy transportation of energy) production can be associated with a unique identifier (e.g., number, name, etc.). The identifier can comprise, for example, a tag, RFID button, etc. In this example, the identifier can be associated with data corresponding to the selected energy producing segment. The data can include, for example, performance data, and the like. Performance data can be associated with on-site management practices, properties data, etc. Some energy production segments can be identified with numbers, letters, shapes, codes, images, RFID tags, bar codes, scannable codes, brands, blockchain data, etc.

Based on the selected segment of energy production, performance data can be obtained 448, which, as described above, may be comprised of on-site practices management data. In an example, the obtained performance data may be used to update the generated model. For example, the obtained performance data may be used as input data that may be processed to determine expected emissions in accordance with embodiments described herein. This can include, for example, identifying 450 performance data variables that may affect emissions calculation. For example, as described, an equation component can model the impact of certain input parameters on an energy product's lifecycle emissions. In this situation, an equation component can be configured to quantify an amount of emissions based on certain performance data, such as on-site management practice data. For example, the impact of wind turbine design, weather conditions, installation energy, system downtime, transmission losses, etc. can be used.

Identifying a data variable associated with at least one equation component of the plurality of equation components can include, for example, identifying data variables associated with equation components determined to impact or otherwise change a level of emissions at least a threshold amount. For example, as described, an equation component can model the impact of certain input parameters on energy production lifecycle emissions.

As such, an equation component can be configured to quantify an amount of emissions based on certain performance data. In a specific example, an equation component can be configured to quantify an amount of emissions based on energy distributions systems relationship with the energy production system. Such a relationship can be represented by at least one equation component. Further, a data variable associated with the relationship can be identified.

The process applies 452 adjustments to identified data variables of equation components to generate 454 an updated model based on identified performance data points, the updated model quantifying an amount of emissions by the selected energy production segment. In one exemplary embodiment, the process applies scaling factors, thresholds, and/or multipliers to model equation components to ensure that an appropriate emissions calculation is obtained based on performance data. The amount and nature of the adjustments may be calculated by the performance data and/or the data point's likely impact on the calculated expected emissions values. Generally, the adjustments may be calculated based on historical performance data and/or in real time or near real time. For example, adjustment parameters can be determined along with a range of potential model values and the baseline value for each parameter. Additionally, in another exemplary embodiment of the adjustment module 126, thresholds may be applied to the model equations (i.e., positive or negative).

The process determines 456 expected emissions by applying adjusted modeling equations to obtain model output. In one exemplary embodiment, the process determines expected emissions by applying a simulation to create expected probability distributions. In one embodiment, the process determines or models the results many times to figure out the range of possible emissions and the likelihood of the actual value being within the range. One exemplary simulation to calculate expected emissions values may be a Monte Carlo simulation wherein the input is random and many simulations are run in order to determine the probabilities of different outcomes. Other simulations may be used as would be apparent to one skilled in the art.

A variety of different outputs may be calculated, including, but not limited to values for greenhouse gas fluxes, product yields, or other metrics, such as: total lifecycle CO₂e emissions, CO₂e emissions for part of the lifecycle, CO₂e absorbed on farm credit, respiratory CO₂e emissions, manure CO₂e emissions, CO₂e emissions from enteric CH₄, CO₂e emissions upstream (upstream emissions are emissions that occur outside of the production process, but are “embedded” in energy or materials that are used in the production process, CO₂e credits, negative CO₂e emissions. In various embodiments, the units of emissions outputs may include, for example, MJ, kWh, barrel, btu, cf, lb, kg, gal, gge, etc.

FIG. 4D below illustrates an exemplary process 460 for estimating emissions during the production of a product, derivative product, or material in accordance with an exemplary embodiment. In this example, the process starts by obtaining 462 historic data from a variety of sources such as academic papers, scientific literature, trade publications, experimental data, etc. In one embodiment, the relevant papers may specifically study the effects of various input parameters on emissions. In the same or other embodiments, the relevant papers may study product characteristics data that may or may not specifically analyze or study product or material production characteristic data and its direct impact on emissions. For example, the historic data may be comprised of, for example, information about different parts of a product or material production system, including, but not limited to performance data, emissions information, economic impact information, etc. Specifically, historic data may include property data, on-site practices management data, etc. Property data generally refers to quantifiable characteristics of a material, substance, energy form, or energy carrier, such as, for example: mass, temperature, pressure, higher heating value, lower heating value, heat of combustion, heat content, energy content, metabolizable energy, density, energy density, melting point, conductivity, resistance, heat of vaporization, current, charge, voltage, electron volts, biochemical composition, protein content, amino acid profile, fat content, lipid content, fatty acid profile, fiber content, energy content, chemical bonds, moisture content, humidity, cellulose content, carbon content, nitrogen content, radiation, conduction, convection, photons, acoustics, Reynolds number, velocity, acceleration, matter, antimatter, or other quantifiable properties. In one embodiment, the historic data may be comprised of human and/or machine-readable information that may be processed, as described in more detail below, to perform additional analysis.

The data can be associated with a plurality of input parameters, where individual input parameters may be associated with equation components that cause a threshold level of change in emissions for at least one emissions producing system. For example, the relevant papers may specifically study the effects of various input parameters on emissions. This can include, for example, the presentation of one or more equation components modeling the effects of various input parameters on emission. Example input parameters include emissions derived from, e.g., property data, on-site practices management data, etc. Property data includes physical, chemical, electrical, nuclear, magnetic, and thermal data. Property data generally refers to quantifiable characteristics of a material, substance, energy form, or energy carrier, such as, for example: mass, temperature, pressure, higher heating value, lower heating value, heat of combustion, heat content, energy content, metabolizable energy, density, energy density, melting point, conductivity, resistance, heat of vaporization, current, charge, voltage, electron volts, biochemical composition, protein content, amino acid profile, fat content, lipid content, fatty acid profile, fiber content, energy content, chemical bonds, moisture content, humidity, cellulose content, carbon content, nitrogen content, radiation, conduction, convection, photons, acoustics, Reynolds number, velocity, acceleration, matter, antimatter, or other quantifiable properties.

Additional data may be obtained from on-site management and operations of oil-and-gas wells, coal mines, photovoltaic systems, wind power systems, refineries, biorefineries, transport systems, distribution systems, or other site-specific operations within a production system. For example, on-site data might be collected for methane leaks from a natural gas operation or carbon dioxide flue gas from a biorefinery. Similar data may be obtained from on-site management and operations of gold mines, lithium recovery ponds, direct air capture machines producing CO2 or other gases, bentonite mines, propylene chemical plants, sodium hydroxide chemical plants, or other site-specific operations within a production system. For example, on-site data might be collected for energy consumption, GHG emissions from, and CO2 collection by a direct air capture CO2 plant.

The input parameters can be associated with equation components. An equation component can model the impact of certain input parameters on emissions for a variety of product and material production systems. That is, an equation component can be configured to quantify an amount of emissions based on particular data. For example, material harvesting can be represented by at least one equation component. In this example, the equation component can model the impact of harvesting the material. In another example, each of one or more product and material production segments can be represented by at least one equation component. In this example, the equation components can model the impact of transporting the product and/or material on the assessment cycle emissions of the product and/or material emission producing system. In another example, storing product and/or material can be represented by at least one equation component.

The process obtains or identifies one or more equation components to generate 464 a model based on the historic data and/or other data such as performance data. The generated model may incapsulate the relationship between input parameters, other data points, and their likely impact on greenhouse gas emissions. The model, including the equation components, can quantify an amount of emissions by material production systems. The model equations for the model can include, for example, equations representing material production segment models such as, for example, material harvesting models, material transportation models, material storage models, operations models, etc. In a specific example, an equation component of the model may quantify how material storage and/or material transportation affects emissions output by a material production system. The model may include equation components based on historic data in one instance, and/or may be based on a variety of different studies and/or practical correlations that may or may not be present in the historic data. In one embodiment, the equation components may be comprised of historic data, on-site practices and protocol data that may be received from one or more other database/sources.

Continuing with the example of a product and/or material-based emissions producing system, a selection of a segment of product and/or material production can be received 466. The segment (e.g., site location build, material harvesting, material storage, material transportation of) production can be associated with a unique identifier (e.g., number, name, etc.). The identifier can comprise, for example, a tag, RFID button, etc. In this example, the identifier can be associated with data corresponding to the selected product and/or material producing segment. The data can include, for example, performance data, and the like. Performance data can be associated with on-site management practices, properties data, etc. Some material production segments can be identified with numbers, letters, shapes, codes, images, RFID tags, bar codes, scannable codes, brands, blockchain data, etc.

Based on the selected segment of product and/or material production, performance data can be obtained 468, which, as described above, may be comprised of on-site practices management data. In an example, the obtained performance data may be used to update the generated model. For example, the obtained performance data may be used as input data that may be processed to determine expected emissions in accordance with embodiments described herein. This can include, for example, identifying 470 performance data variables that may affect emissions calculation. For example, as described, an equation component can model the impact of certain input parameters on a product and/or material assessment cycle. In this situation, an equation component can be configured to quantify an amount of emissions based on certain performance data, such as on-site management practice data. For example, the impact of geological formation, mining equipment, mining fuels, transport distance, transport equipment, transport fuels, etc. can be used.

Identifying a data variable associated with at least one equation component of the plurality of equation components can include, for example, identifying data variables associated with equation components determined to impact or otherwise change a level of emissions at least a threshold amount. For example, as described, an equation component can model the impact of certain input parameters on product and/or material production lifecycle emissions.

As such, an equation component can be configured to quantify an amount of emissions based on certain performance data. In a specific example, an equation component can be configured to quantify an amount of emissions based on product and/or material transportation systems relationship with the product and/or material production systems. Such a relationship can be represented by at least one equation component. Further, a data variable associated with the relationship can be identified.

The process applies 472 adjustments to identified data variables of equation components to generate 474 an updated model based on identified performance data points, the updated model quantifying an amount of emissions by the selected product and/or material production segment. In one exemplary embodiment, the process applies scaling factors, thresholds, and/or multipliers to model equation components to ensure that an appropriate emissions calculation is obtained based on performance data. The amount and nature of the adjustments may be calculated by the performance data and/or the data point's likely impact on the calculated expected emissions values. Generally, the adjustments may be calculated based on historical performance data and/or in real time or near real time. For example, adjustment parameters can be determined along with a range of potential model values and the baseline value for each parameter. Additionally, in another exemplary embodiment of the adjustment module 126, thresholds may be applied to the model equations (i.e., positive or negative).

The process determines 476 expected emissions by applying adjusted modeling equations to obtain model output. In one exemplary embodiment, the process determines expected emissions by applying a simulation to create expected probability distributions. In one embodiment, the process determines or models the results many times to figure out the range of possible emissions and the likelihood of the actual value being within the range. One exemplary simulation to calculate expected emissions values may be a Monte Carlo simulation wherein the input is random and many simulations are run in order to determine the probabilities of different outcomes. Other simulations may be used as would be apparent to one skilled in the art.

A variety of different outputs may be calculated, including, but not limited to values for greenhouse gas fluxes, product yields, or other metrics, such as: total lifecycle CO₂e emissions, CO₂e emissions for part of the lifecycle, CO₂e absorbed on farm credit, respiratory CO₂e emissions, manure CO₂e emissions, CO₂e emissions from enteric CH₄, CO₂e emissions upstream (upstream emissions are emissions that occur outside of the production process, but are “embedded” in energy or materials that are used in the production process, CO₂e credits, negative CO₂e emissions. In various embodiments, the units of emissions outputs may include, for example, t, lb, kg, m3, cf, carat, density, etc.

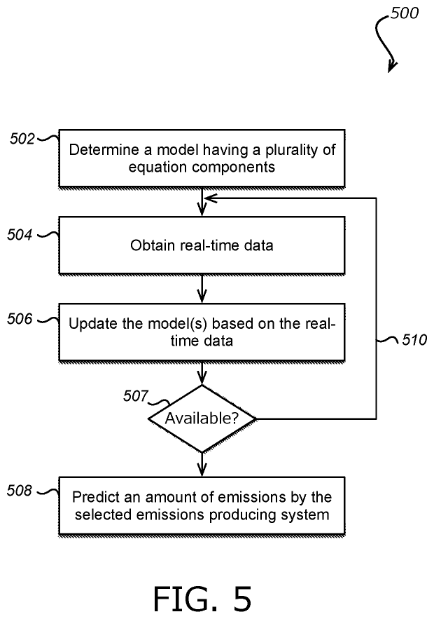

FIG. 5 illustrates an exemplary process 500 for updating a model using real-time data in accordance with various embodiments. In this example, a model comprising a plurality of equation components is determined 502. The model can include at least one of a product-centric models, animal-centric models, crop-centric models, energy production-centric models, material-centric models, combination models, and the like. Real-time data is obtained 504. The real-time data can be obtained using one or more sensors. The sensors can include, for example, devices that produce a change in output dependent on input stimuli, optical sensors, mechanical sensors, magnetic sensors, electrical sensors, electrochemical sensors, image or video recording devices to capture image data, electronic scales to capture weight data, electronic feeders to capture weight data, temperature and thermal sensors to capture temperature and energy data, pressure sensors, humidity sensors to capture humidity data, gas sensors to capture gas data, movement sensors to capture movement data, pH sensors, GPS data, body composition sensors, health measurements, ultrasound measurements, animal recognition and identification sensors, sonic sensors, flowrate sensors, sensors of stress, strain, position, particles, or force, genetic sensors, chemical sensors for concentration or identity of a substance, element, molecule, or compound, biosensors for biological measurements of pathogens, enzymes, cells, hormones, pressures, pH, elements (Ca, Mg, etc.), protein, carbohydrates, etc., radar, spectrophotometry, spectroscopy, calorimetry, chromatography, microscopy, X-ray, gravitational sensors, sub-atomic particle sensors, RFID sensors, radioactivity sensors, light sensors, etc.

The sensors can obtain data related to animal performance data such as animal consumption data, emissions data, and animal behavior data. In an example, the sensors can be used to monitor automatically and continuously the consumption, emissions, and the behavior of individual animals. The data can be used to predict and determine a variety of conditions relating to health, feed efficiency, animal welfare, performance, and production efficiency enabling determination of individual animal performance on different rations, response to medications, response to feed supplements, response to minerals and trace minerals, response to growth promoting substances, prediction of carcass quality, which can be used to determine greenhouse gas and manure excretion. In another example, the sensors can be used to monitor automatically and continuously aspects associated with product-centric models, crop-centric models, energy production-centric models, and the like. For example, the sensors can be used to monitor automatically and continuously farms, elevators, mills, food processing facilities, energy producing processes (e.g., wells, mines, refineries, etc.), material processes (e.g., wells, mines, ships, trucks, refineries, chemical processing plants, etc. It should be noted that based on the type of model additional or fewer sensors may be utilized. Similarly, sensors can be used to measure and quantify aspects of crop, energy, material, and other product production systems.

The equation components can be updated 506 to generate an updated model based on the real-time data. For example, input parameters of the model can be dynamically selected based on available data. This can include, for example, adding or removing one or more input parameters based on the presence (or absence) of the animal performance data, crop data, energy data, and/or material data. For example, as described, equation components can be identified based on input parameters and the equation components can be adjusted based on real-time data. In this example, the equation components can include equations to weaning weight (WnWt), yearling weight (YrWt), calf death loss rate (CalDL), dry matter intake (DMI), average daily grain (ADG), cow death loss rate (CowDL), carcass weight (CarcWt), etc. In another example, this can include adding or removing one or more input parameters based on the presence (or absence) of product, crop, energy, material, and/or product data. In certain embodiments, the equation components include input parameters associated with one or more of emissions producing systems.

A determination can be made 507 whether data is available. In the situation no new data is available, an amount of emissions by an emissions producing system can be predicted 508 by evaluating the updated model on the emissions data and the performance data. In various embodiments, the model can be updated based on available data for one or more emissions producing systems. Additionally or alternatively, the emissions data can be compared to an emissions threshold. In the situation the emissions data represents a level of emissions that does not satisfy the emissions threshold, control instructions including, for example, computer readable instructions, can be generated to control appliances (e.g., on-site appliances) to achieve emissions goals or thresholds. This can include, for example, control instructions including, for example, computer readable instructions, can be to alter the feed formulation, grazing areas, manure management, power management, food processing facilities, energy producing processes, material processes, etc. In the situation new data is available (e.g., data associated with crop, energy, material, product emissions production systems), the process can repeat 510 to update the model.

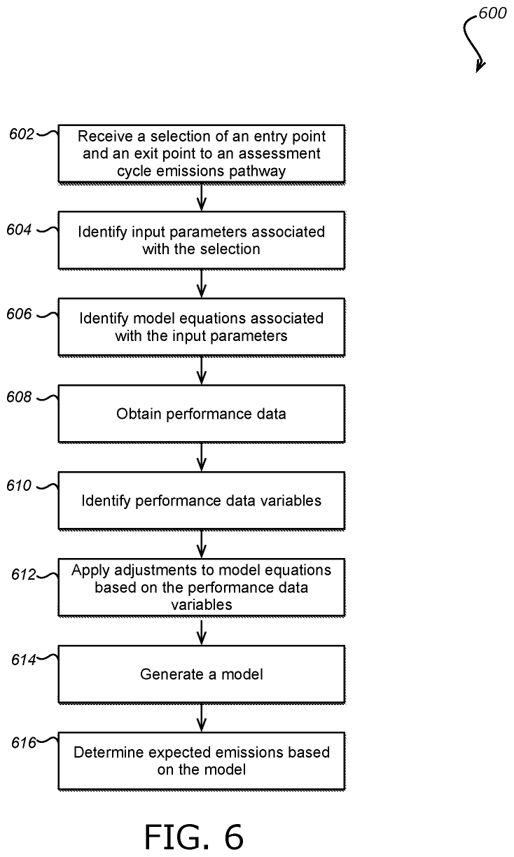

In an embodiment, the model may be updated based on an entry point (e.g., a start date) and exit point (e.g., an end date) of the model. For example, FIG. 6 illustrates example 600 for updating a model based on assessment cycle emissions pathways in accordance with various embodiments. As described, a model comprises a plurality of model equations. One or more model equations can be associated with a segment of one or more emission producing systems. In an example, the one or more model equations can be associated with a segment of an animal's assessment cycle emissions, a crops assessment cycle emissions, energy production assessment cycle emissions, material production assessment cycle emissions, product assessment cycle emissions, and the like.

In an embodiment, an animal's assessment cycle emissions can be the amount of emissions by the animal or group of animals during their lifetime or a segment of their lifetime. For example, the animal's assessment cycle emissions can include a plurality of assessment emission pathways. An example assessment emission pathway can include, a cow-calf assessment emission pathway, a backgrounding assessment emission pathway, a grass finished assessment emission path, etc. Another example assessment emission pathway can include cow-calf, backgrounding, and feedlot. Yet another example assessment emission pathway can include cow-calf and direct entry to feedlot. The model can output different combinations of emissions (e.g., emissions from segments of pathways such as the feedlot segment) or the model may output the total emissions for any one of the lifecycle emissions pathways.

In an embodiment, a crop's assessment emission pathway can be the amount of emissions from production of the crop during the complete production cycle of the crop or a segment of the production cycle. In an embodiment, energy production assessment emissions can be the amount of emissions from production of the energy or an amount of energy during the complete production cycle of the energy or a segment of the production cycle. In an embodiment, material production assessment emissions can be the amount of emissions from production of the material during the complete production cycle of the material or a segment of the production cycle. In an embodiment, product production assessment emissions can be the amount of emissions from production of the material during the complete production cycle of the product or a segment of the production cycle. As described, the model can output different combinations of emissions (e.g., emissions from segments of pathways such as the feedlot segment) or the model may output the total emissions for any one of the lifecycle emissions pathways.

A selection of an entry point and an exit point is received 602. The entry point can correspond to an assessment cycle emissions pathway, where the pathway can be associated with one or more model equations. For example, the selection can indicate an assessment emissions pathway that considers one or more product-specific and/or production-specific emissions models. As described, the models can include cow-calf models, backgrounding modes, grass finished models, models associated with the production of crops, energy carriers, materials, products, derivative products, and the like. In another example, the selection may include an indication of one or more particular segments of pathways. For example, the selection may indicate the grass finished segment, energy storage segment, crop transport segment, etc. The model can include a model to quantify emissions from a derivative product. Said another way, a model configured to quantify the emissions from one product can be adapted to quantify the emissions from a derivative product.

In accordance with embodiments described herein, historic data associated with the selected assessment emissions pathway can be obtained and a selection of an emissions pathway can be received. In an example, this may include a selection of an animal or a group of animals, a crop or a group of crops, production or segment of production or energy, material, a product, etc.

Input parameters from historic data associated with the selected lifecycle emissions pathway can be identified 604.

Model equations associated with the input parameters can be identified 606. For example, as described herein, the equation components can be based on input parameters that model the impact of certain characteristics on the assessment cycle emissions. This can include, in an example, equation components that model the impact of the type of fertilizer being applied to the grass pasture (e.g., organic fertilizer or inorganic fertilizer) on an animal's lifecycle emissions or electricity from fossil fuels versus renewable energy; operation processes at farms, elevators, mills, food processing facilities; energy producing processes (e.g., wells, mines, refineries, etc.); material processes (e.g., wells, mines, ships, trucks, refineries, chemical processing plants, etc.

Performance data, which may be comprised of expected progeny performance data, properties data, genetic data, phenotypic data, on-site practices management data, etc. can be obtained 608 and performance data variables that may affect emissions can be identified 610. In accordance with embodiments described herein, adjustments to the model equations can be applied 612 based on the performance and other appropriate data. A model can be generated 614. The model includes the model equations. In this example, model generation can include selecting equation components based on the historic data and performance data. In other examples, model generation can include selecting equation components based on the historic data to generate a model, and updating the model based on performance data. In certain embodiments, selecting equation components can be based on historic data and/or performance data. In any situation, a model can be iteratively updated in real-time or other time interval or event detection based on real-time data as described herein.

The model equations can be organized in one or more groups. The one or more groups can be arranged in a hierarchical structure. The structure can include one or more nodes. The path from node to node or node to a base level (i.e., the entry points and exit points) can be considered an assessment cycle emission pathway. In an example, the assessment cycle emission pathway corresponds to a segment of the assessment cycle of one or more animals and can use a particular set of model equations to determine emissions output by the one or more animals. In another example, the assessment cycle emission pathway corresponds to a segment of a crops assessment cycle emissions, energy production assessment cycle emissions, material production assessment cycle emissions, product assessment cycle emissions, and the like. Thereafter, expected emissions for one or more or a combination of emission producing systems can be determined 616 by evaluating the model. As described, this can include applying the model to obtain a model output, including, but not limited to values for greenhouse gas fluxes, product yields, or other metrics, such as: total lifecycle CO₂e emissions, CO₂e emissions for part of the lifecycle, CO₂e absorbed on farm credit, respiratory CO₂e emissions, manure CO₂e emissions, CO₂e emissions from enteric CH₄, CO₂e emissions upstream (upstream emissions are emissions that occur outside of the production process, but are “embedded” in energy or materials that are used in the production process), CO₂e emissions from manure N₂O, CO₂e directly emitted on farm, CO₂e emissions from soil N₂O, CO₂e emissions from soil N₂O, CO₂e emissions from soil N₂O, CO₂e emissions from manure CH₄, CO₂e sequestered in soil or other media, CO₂e credits, negative CO₂e emissions, CO₂e sequestration fluxes, carcass weight yield, by-product yields, manure yield, etc. Output metrics can include a variety of measures, such as, but not limited to: kg CO₂e/kg carcass weight, kg CO₂e/head, etc.

In accordance with various embodiments, certification module (e.g., certification module 308) assigns one or more certification(s) if the expected emissions are calculated to be above, below, or otherwise satisfy a designated threshold. In one embodiment, the certification module 308 indicates the amount of greenhouse gas emissions that an emissions producing system (e.g., emissions from an animal, crop, energy, material or other product) has emitted and/or expected to emit in accordance with the calculation system described herein. Certifications could include transaction related to product labeling, emissions limits (caps), emissions trades, emissions taxes, emissions offset (i.e., credits), other emissions transactions, etc.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

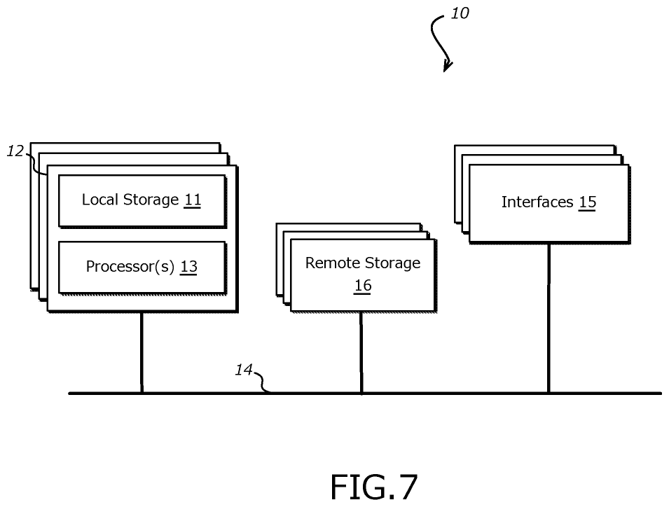

Referring now to FIG. 7, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASIC s), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 7 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

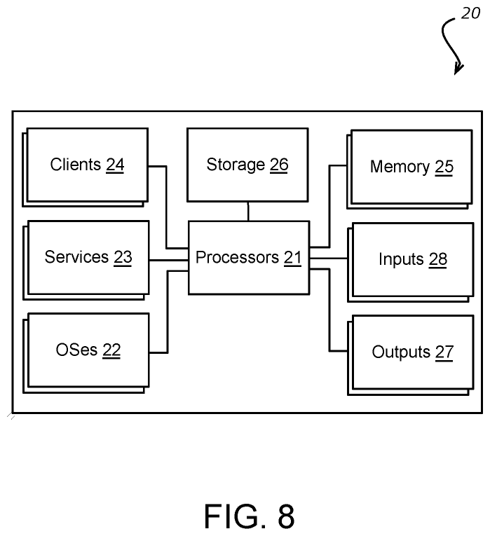

In some embodiments, systems may be implemented on a standalone computing system. Referring now to FIG. 8, there is shown a block diagram depicting a typical exemplary architecture of one or more embodiments or components thereof on a standalone computing system. Computing system 20 includes processors 21 that may run software that carry out one or more functions or applications of embodiments, such as for example a client application 24. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications 24. Services 23 may for example be WINDOWS™ services, user-space common services in a Linux environment, or any other type of common service architecture used with operating system 22. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described with respect to FIG. 7). Examples of storage devices 26 include flash memory, magnetic hard drive, CD-ROM, and/or the like.

In some embodiments, systems may be implemented on a distributed computing network, such as one having any number of clients and/or servers.

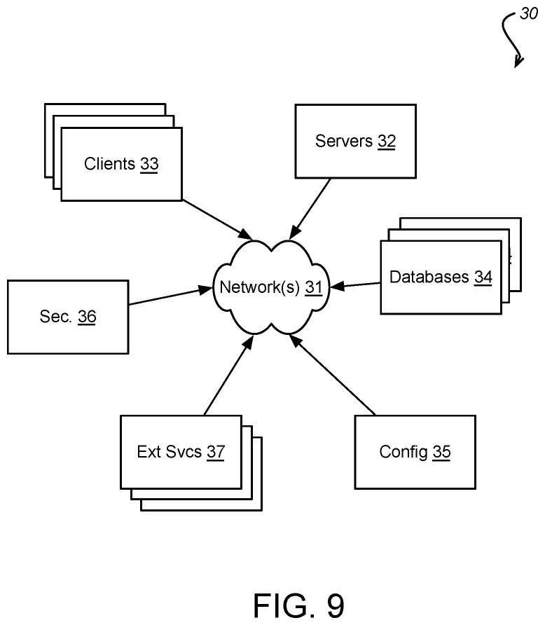

Referring now to FIG. 9, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 8. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications 24 are implemented on a smartphone or other electronic device, client applications 24 may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system.

Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

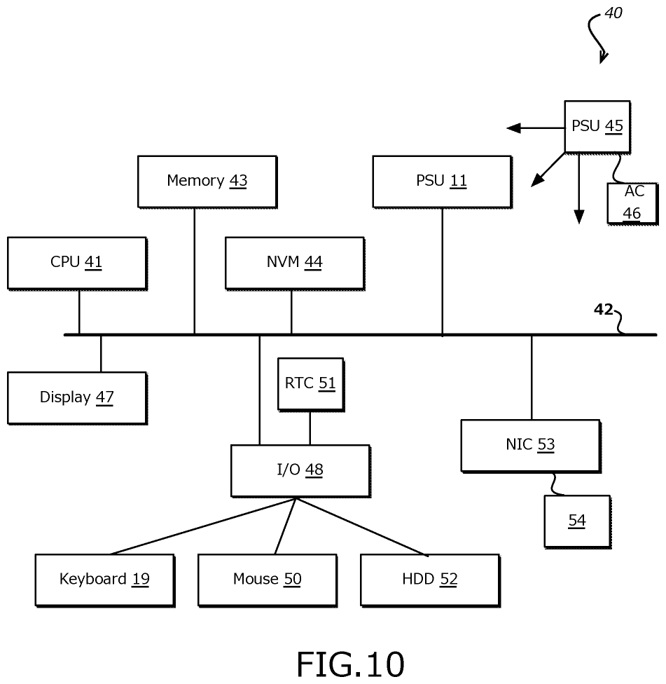

FIG. 10 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48 (including, e.g., keyboard 19, mouse 50, HDD 52, etc.) and network interface card (NIC) 53. I/O unit 48 may, typically, be connected to keyboard 19, pointing device 52, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined,

such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or.” For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

1. A computing system for generating emissions models, the computing system comprising:
a computing device processor; and
a memory device including instructions that, when executed by the computing device processor, enables the computing system to:
obtain, by the computing device processor of the computing system, historic product data from a plurality of different disaggregated sources,
identify, by the computing device processor of the computing system, a plurality of equation components based on the historic product data, individual equation components configured to quantify an amount of emissions,
generate, by the computing device processor of the computing system, an emissions model comprising the plurality of equation components, the emissions model quantifying a total amount of emissions by a group of products for an emissions lifecycle of the group of products, wherein the emissions lifecycle includes a plurality of potential assessment emissions pathways,
receive a selection of a product associated with the group of products to identify a selected product, the product associated with a unique identifier identifying the selected product,
obtain in real-time from a database, by the computing device processor of the computing system, wherein the database is comprised of information obtained by at least one sensor of a plurality of sensors monitoring the selected product, performance data associated with the unique identifier of the selected product,
identify, by the computing device processor of the computing system, one or more data variables associated with at least one equation component of the plurality of equation components of the emissions lifecycle based on the performance data, and
apply, by the computing device processor of the computing system, at least one adjustment to the at least one equation component to generate a product-centric emissions model, the product-centric emissions model quantifying an amount of emissions by the selected product during an emissions assessment cycle of the selected product.

2. The computing system of claim 1, wherein the instructions, when executed by the computing device processor, further enables the computing system to:
receive a selection of a pathway from the plurality of potential assessment emissions pathways for the selected product, the pathway comprising an entry point corresponding to a start date and an exit point corresponding to an end date,
wherein the product-centric emissions model is based on the pathway.

3. The computing system of claim 2, wherein the instructions, when executed by the computing device processor, further enables the computing system to:
identify, by the computing device processor of the computing system, equation components associated with the pathway,
wherein the product-centric emissions model is based on the equation components.

4. The computing system of claim 1, wherein the instructions, when executed by the computing device processor, further enables the computing system to:
determine, by the computing device processor of the computing system, the amount of emissions by the selected product during the emissions assessment cycle of the selected product by evaluating the product-centric emissions model on the historic product data and the performance data.

5. The computing system of claim 1, wherein the amount of emissions by the selected product is for a particular assessment emissions pathway.

6. The computing system of claim 1, wherein the instructions, when executed by the computing device processor, further enables the computing system to:
display, for the selected product associated with the unique identifier, in a graphical user interface, one or more views of the amount of emissions during the emissions assessment cycle.

7. The computing system of claim 1, wherein the instructions, when executed by the computing device processor, further enables the computing system to:
associate at least one certification, label, emissions limit/cap, emissions trade, emissions offset/credit, or other emissions transaction with the selected product based on the amount of emissions by the selected product during the emissions assessment cycle of the selected product.

8. The computing system of claim 7, wherein the at least one certification or other transaction indicates the amount of emissions that the product has emitted or is expected to emit.

9. The computing system of claim 1, wherein the instructions, when executed by the computing device processor, further enables the computing system to:
iteratively update the product-centric emissions model based on additional data from the plurality of sensors.

10. The computing system of claim 9, wherein a machine learning technique is utilized to iteratively update the product-centric emissions model.

11. The computing system of claim 1, wherein a machine learning technique is utilized to generate the emissions model.

12. The computing system of claim 1, wherein the plurality of sensors includes at least one of a camera, a scale, a ruler, a timer, a feeder, a temperature sensor, a pressure sensor, a flow meter, an electrical sensor, a radiation sensor, a gas sensor, a liquid sensor, a humidity sensor, a movement sensor, a global positioning sensor (GPS), a soil composition sensor, a pH sensor, a body composition sensor, a health sensor, animal identification sensor, crop identification sensor, energy carrier identification sensor, material identification senso, facial identification sensor, biomedical sensor, an x-ray sensor, nuclear magnetic resonance sensor, or an ultrasound sensor, and wherein the performance data includes expected progeny performance data, expected progeny differences data, genetic data, phenotypic data, properties data and on-site practices management data associated with the selected product.

13. The computing system of claim 1, wherein the instructions, when executed by the computing device processor, further enables the computing system to:
generate control instructions to control an appliance to alter at least one task affecting the amount of emissions by the selected product during the emissions assessment cycle of the selected product.

14. A computer-implemented method for generating product-centric emissions models, comprising:
obtaining, by a computing device processor, historic product data from a plurality of different disaggregated sources,
identifying, by the computing device processor, a plurality of equation components based on the historic product data, individual equation components configured to quantify an amount of emissions,
generating, by the computing device processor, an emissions model comprising the plurality of equation components, the emissions model quantifying a total amount of emissions by a group of products for an emissions assessment cycle of the group of products, wherein the emissions assessment cycle includes a plurality of potential assessment emissions pathways,
receiving a selection of a product associated with the group of products to identify a selected product, the product associated with a unique identifier identifying the selected product,
obtaining in real-time from a database, by the computing device processor, wherein the database is comprised of information obtained by at least one sensor of a plurality of sensors monitoring the selected product, performance data associated with the unique identifier of the selected product,
identifying, by the computing device processor, one or more data variables associated with at least one equation component of the plurality of equation components of the emissions assessment cycle based on the performance data, and
applying, by the computing device processor, at least one adjustment to the at least one equation component to generate a product-centric emissions model, the product-centric emissions model quantifying an amount of emissions by the selected product during the emissions assessment cycle of the selected product.

15. The computer-implemented method of claim 14, further comprising:
receiving a selection of a pathway from the plurality of potential assessment emissions pathways for the selected product, the pathway comprising an entry point corresponding to a start date and an exit point corresponding to an end date,
wherein the product-centric emissions model is based on the pathway.

16. The computer-implemented method of claim 15, further comprising:
identifying, by the computing device processor, equation components associated with the pathway,
wherein the product-centric emissions model is based on the equation components.

17. The computer-implemented method of claim 14, further comprising:
determining, by the computing device processor, the amount of emissions by the selected product during the emissions assessment cycle of the selected product by evaluating the product-centric emissions model on the historic product data and the performance data.

18. The computer-implemented method of claim 17, further comprising:
determining, by the computing device processor, an emissions offset based on the total amount of emissions by the group of products and the amount of emissions by the selected product.

19. The computer-implemented method of claim 14, wherein the database comprises a blockchain database.

20. A non-transitory computer readable storage medium storing instructions that, when executed by a computing device processor of a computing system, causes the computing system to:
obtain, by the computing device processor, historic product data from a plurality of different disaggregated sources,
identify, by the computing device processor, a plurality of equation components based on the historic product data, individual equation components configured to quantify an amount of emissions,
generate, by the computing device processor, an emissions model comprising the plurality of equation components, the emissions model quantifying a total amount of emissions by a group of products for an emissions assessment cycle of the group of products, wherein the emissions assessment cycle includes a plurality of potential assessment emissions pathways,
receive a selection of a product associated with the group of products to identify a selected product, the product associated with a unique identifier identifying the selected product,
obtain in real-time from a database, by the computing device processor, wherein the database is comprised of information obtained by at least one sensor of a plurality of sensors monitoring the selected product, performance data associated with the unique identifier of the selected product,
identify, by the computing device processor, one or more data variables associated with at least one equation component of the plurality of equation components of the emissions assessment cycle based on the performance data, and
apply, by the computing device processor, at least one adjustment to the at least one equation component to generate a product-centric emissions model, the product-centric emissions model quantifying an amount of emissions by the selected product during the emissions assessment cycle of the selected product.
